(19) United States
(12) Patent Application Publication
(10) Pub. No.: US 2021/0205244 A1
(43) Pub. Date: Jul. 8, 2021

(54) PREVENTION AND/OR TREATMENT OF CHRONIC FATIGUE SYNDROME

(71) Applicant: BOHNE ASKØY AS, Hauglandshella (NO)

(72) Inventors: Øyvind BOHNE, Hauglandshella (NO); Victoria BOHNE, Hauglandshella (NO)

(21) Appl. No.: 16/463,679

(22) PCT Filed: Nov. 23, 2017
(86) PCT No.: PCT/NO2017/000032

§ 371 Date: May 23, 2019

(30) Foreign Application Priority Data
Nov. 23, 2016 (NO) 20161866

(51) Int. Cl. A61K 31/194; A61K 31/385; A61K 31/455; A61K 31/51; A61K 31/525; A61K 47/26

(52) U.S. Cl. CPC A61K 31/194; A61K 31/385; A61K 47/26; A61K 31/51; A61K 31/525; A61K 31/455

(57) ABSTRACT

A composition for the prevention and/or treatment of chronic fatigue syndrome (CFS)/myalgia encephalomyelitis (ME)/systemic exertion intolerance disease (SEID) is described. Also described is a method for diagnosis of a patient with chronic fatigue syndrome (CFS)/myalgia encephalomyelitis (ME)/systemic exertion intolerance disease (SEID).

### FIELD OF INVENTION

The present invention relates to a composition for the prevention and/or treatment of chronic fatigue syndrome (CFS)/myalgia encephalomyelitis (ME)/systemic exertion intolerance disease (SEID) is described. The invention also relates to a method for diagnosis of a patient with chronic fatigue syndrome (CFS)/myalgia encephalomyelitis (ME)/systemic exertion intolerance disease (SEID).

### BACKGROUND OF THE INVENTION

Chronic fatigue syndrome (CFS) is a long-term illness with a wide range of symptoms. CFS is also known as ME, which stands for myalgia encephalomyelitis. In 2015, the Institute of Medicine proposed a new name for this syndrorn—systemic exeruon intolerance disease (SEID).

The cause of chronic fatigue syndrome (CFS/ME/SEID) is unknown and the risk factors are not clearly understood.

The symptoms and signs of chronic fatigue syndrome (CFS/ME/SEID) are relatively specific; chronic severe fatigue for at least five to six months not caused by a diagnosable disease and at least four other specific symptoms such as cognitive impairment, muscle and/or joint pains, headaches, tender lymph nodes, sore throat, unrefreshing sleep, and malaise after exercise.

We will in the present application use the abbreviation “CFS/ME/SEID)” of the medical indications term Chronic fatigue syndrome (CFS), myalgia encephalomyelitis (ME) and systemic exertion intolerance disease (SEID).

The inventors of the present invention have surprisingly found that oxalic acid or derivatives or salts thereof have a curative effect on CFS/ME/SEID. We do not know the exact mechanisms of the active compounds but key metabolic pathways are modulated by the addition of the compounds of the present invention.

A number of voluntary persons, with and without CFS/ME/SEID have been tested, and the compounds of the present invention show a remarkable improvement of many biological parameters and symptoms.

### SUMMARY OF THE INVENTION

A first aspect of the present invention relates to a composition for the prevention and/or treatment of chronic fatigue syndrome (CFS)/myalgia encephalomyelitis (ME)/systemic exertion intolerance disease (SEID), comprising administering to a patient in need thereof a pharmaceutical or nutritional composition comprising oxalate or oxalic acid, or a salt prodrug, derivative or metabolite thereof.

In an embodiment comprises the composition an oxalic compound of the formula

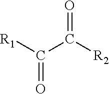

where;
R₁═OH and R₂═OH is oxalic acid, or
R₁═H₂N and R₂═ONa is sodium oxamate, or
R₁═H₂N and R₂═OK is potassium oxamate, or
R₁═H₂N and R₂═CaO is calcium oxamate, or
R₁═O⁻ or R₂═OH is hydrogenoxalate, or
R₁═K⁺O⁻, R₂═OH is potassium hydrogenoxalate, or
R₁═O⁻ and R₂═O⁻ is oxalate, or
R₁═NaO and R₂═NaO is sodium oxalate, or
R₁═CaO and R₂═CaO is calcium oxalate

In an embodiment comprises the composition a compound selected from the group consisting of magnesium oxalate, potassium oxalate, oxalic acid anhydrous, oxalic acid dihydrate, lithium oxalate, cesium oxalate, oxaloacetic acid, lithium oxamate, cesium oxamate, magnesium oxamate, caesium oxalate, beryllium oxalate, potassium oxalate, oxalic acid anhydrous, oxalix acid dyhydrate, lithium oxalate, sodium oxalate, thallium (I) oxalate, uranyl oxalate, gallium oxalate, gold oxalate, magnesium oxalate, mercury (II) oxalate, manganese oxalate, nickel oxalate, barium oxalate, silver oxalte, iron (II) ferrous oxalate, scandium oxalate, cadmium oxalate, and calcium oxalate.

In an embodiment comprises the composition lipoic acid, preferably alpha-lipoic acid (ALA).

In an embodiment comprises the composition thiamine, B₁. In an embodiment comprises the composition niacin, B₃. In an embodiment comprises the composition In an embodiment comprises the composition Riboflavin, B₂.

In an embodiment comprises the composition a sugar.

In an embodiment is the sugar one or more sugars selected from the group consisting of sucrose, glucose, fructose, maltose and lactose.

In an embodiment comprises the disorder in addition to the fatigue syndrome one or more symptoms or disorders selected from, acidosis, myalgia encephalomyelitis (ME), fibromyalgia, ageing, sleep quality, and physical and cognitive activity.

In an embodiment is the disorder selected from;
i) Heart functioning failure as acute cardiac patients with acute coronary syndrome, cardiogenic shock, cardiac arrest with hyperlactemia (high lactate levels and poore lactate clearance), high resting heart rate.
ii) Ortostatic intolerance, essential hypertention, hypotention
iii) All metabolic disorders where occumulation of lactate and its delated or poor excretion/clearance are main cause of the death;
iv) All normal physiological and medical conditions where occumulation of lactate and its delated or poor excretion/clearance are present;
v) All cognitive disorders, which are caused by or causing the occumulation of lactate and its delated or poor excretion/clearance;
vi) All medical conditions where functioning of the organs, tissues and systems are corrupted as the result of the decreased energy production and occumulation of lactate and its delated or poor excretion/clearance as listed in pkt. 1-5.
vii) As claimed in pkt. 6 and in addition to mentioned all symptoms in Tab.3 also astma provoked by physical activity (extreme sports) and/or cold.
viii) Sleep distorbansy, especially insomnia
ix) Irritable Bowel syndrome
x) Fatigue caused by chemotherapy.

The invention relates in a second aspect to a method for diagnosis of a patient with chronic fatigue syndrome (CFS)/myalgia encephalomyelitis (ME)/systemic exertion intolerance disease (SEID), wherein an abnormal level of lactate or lactate pattern in the patient's blood indicates that the patient has CFS/ME/SEID.

In an embodiment is an abnormal level of lactate is a lactate level above a reference value from population not suspected for ME/SEID/CFS.

In an embodiment is an abnormal level of lactate a lactate level in blood above 2.0 mmol/L.

In an embodiment is an abnormal level of lactate measured as a Total lactate Load measured in the patient's blood, and wherein the Total lactate Load measured is above a reference value determined from population not suspected for ME/SEID/CFS.

In an embodiment is the Total Lactate Load above 213±46 mmol/L lactate*min.

In an embodiment is an abnormal lactate pattern measured as Instantaneous Lactate Fluctuation (ILF), and wherein a Instantaeous Lactate Fluctuations within 5 minutes are more than |1.5 mmol/L|, meaning that difference between two consecutive measurements should be higher than 1.5 mmol/L, independently whether it is an increasing or decreasing trend.

In an embodiment is said abnormal level of lactate or lactate pattern measured as; (1) abnormal Basal Housekeeping Lactate Levels (BHLL) in a patient, who is not moving and thus without any contribution from the muscle job or anaerobe threshold, and (2) Instantaneous Lactate Fluctuations (ILF) during given time visualized by lactatogram; (3) and Total Lactate Load (TLL) of the capillary blood calculated as Area Under the Curve (AUC) for measurements during 155 minutes.

In an embodiment is an abnormal level of lactate or lactate pattern measured in extremities.

In an embodiment is the diagnosis of CFS/ME/SEID determined with physiological and cognitive stimuli (efforts) performs as following and the given time is counted from start of reading (time point zero):
Test person lying on the bed in good ventilated room with comfortable ambient temperature in 60 minutes prior to first stimuli (−60 min);
During first 50 minutes measurements of capillary lactate taken each 5^{th} minutes (−60 min to −50 min).
Last 10 minutes before reading the test person lying and relaxing as much as possible (−60 min to 0 min);
Measurement is taken just before reading (0 min);
Test person reads the standardized text in mother or English language, which is not adapted for reading disabilities (by script, colour or contrast) for 10 minutes (10 min);
Measurement is taken (10 min);
Test person continue to read for additionally 5 minutes (15 min);
Measurement taken and test person relaxing for next 10 minutes (15 min).
Light meal of banana, bread toast or glass of water consumed during this period (15 min-25 min);
Measurement is taken (25 min);
Test person relaxing for the next 10 minutes before next measurement is taken (35 min);
Totally 8 measurements taken with 5 minutes intervals during following period of time (35 min to 75 min);
Last two measurements are taken with 10 minutes intervals (at 85 min and 95 min);
Right after that, test person exposes the hallux on the left and right foot; and symmetrical fingers on each hand for finally measurements of the Lactate in Extremities (time counting is not necessarily).
Total time for the test is 155 minutes, wherein 60 minutes prior to reading and 95 minutes after.
Lactatogram data set consisting of 26 consecutive measurements.
Lactate in Extremities consist of 4 measurements in each extremity.

### EXPERIMENTAL SECTION

The experiments conducted and the results obtained will be described in the examples below with reference to the figures;

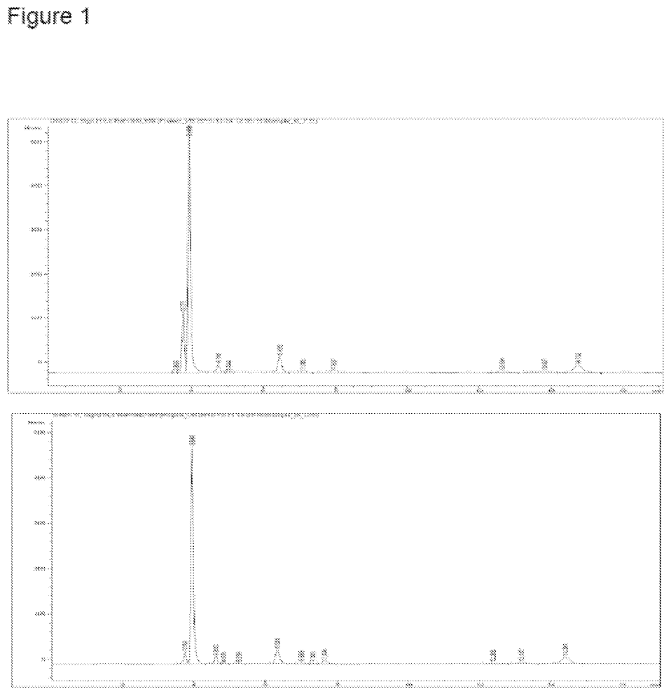

FIG. 1 shows a HPLC chromatogram of Freshly made Active drinkable (above) and freshly Quenched drinkable (below).

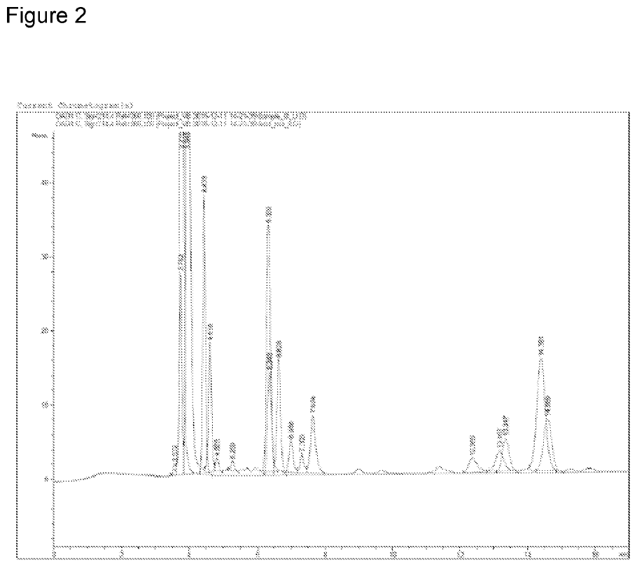

FIG. 2 shows overlaid chromatograms of freshly quenched drinkable and Acid mix.

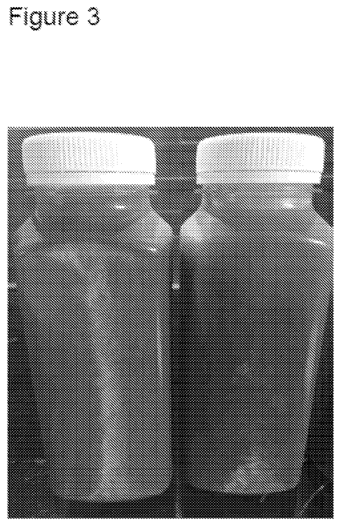

FIG. 3 is a photo of spontaneous formed precipitate under the storage at 4-7° C. in HPP-treated active drinkable.

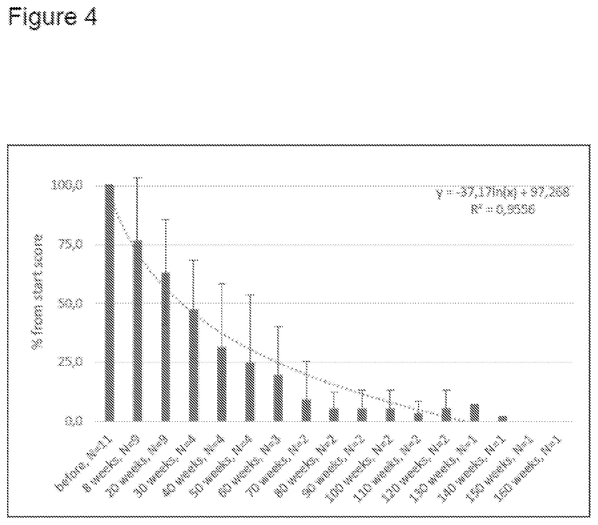

FIG. 4. Mean (+/−S.D.) of remaining symptoms as % from start individual score on the List of Canadian Criteria achieved within a given time with self-treatment by (N) persons.

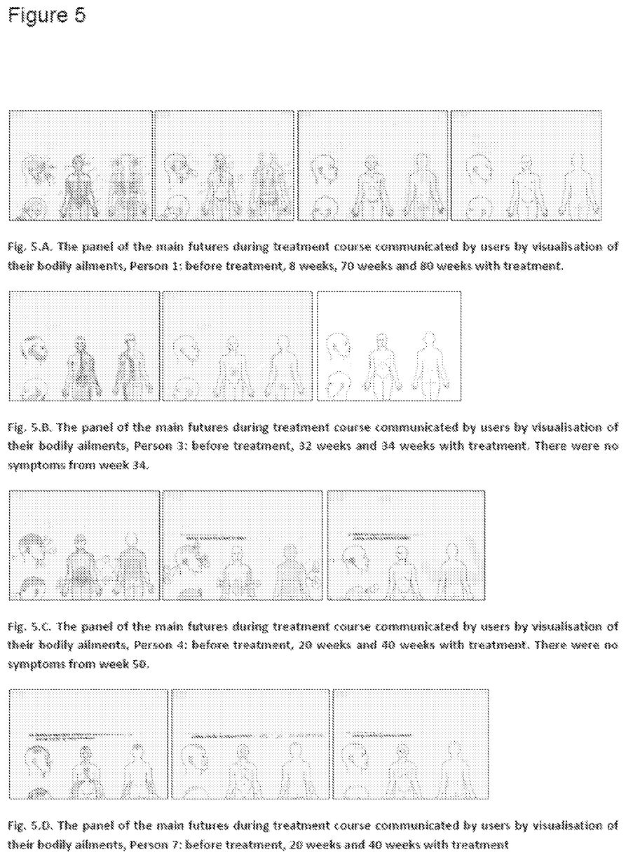

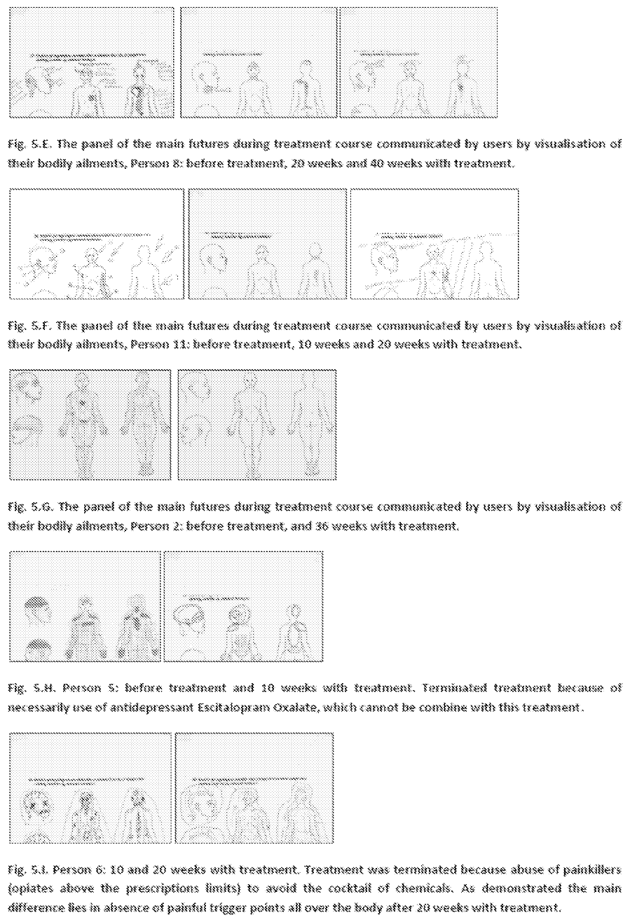

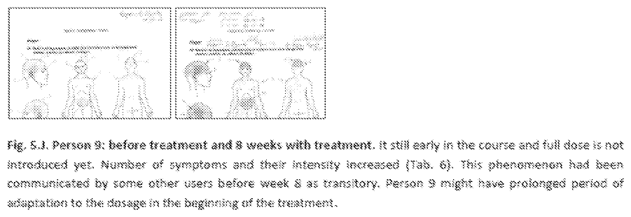

FIG. 5 shows panels of main futures during treatment course communicated by user by visualization of their bodily ailments.

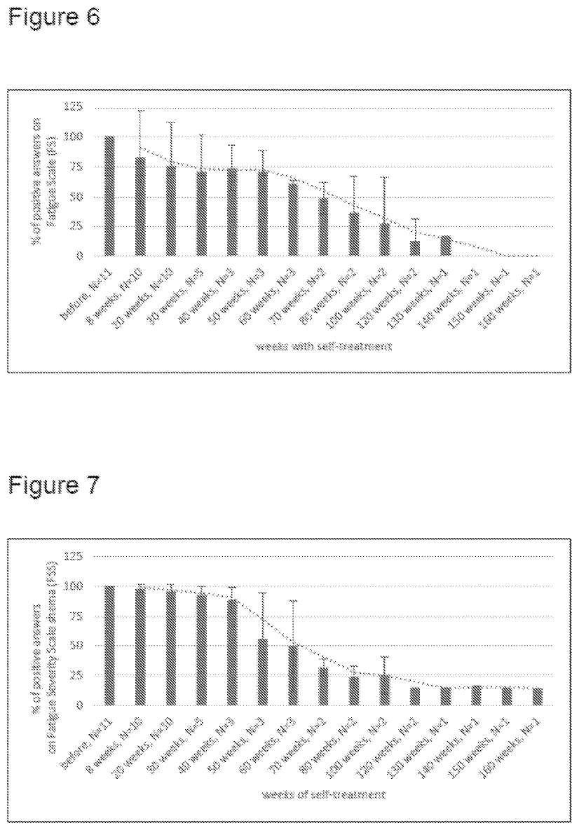

FIG. 6. Mean (+/−S.D.) of remaining score on Fatigue Scale schema (FS) during a self-treatment course in % from individual start score.

FIG. 7. Mean (+/−S.D.) of remaining score on Fatigue Severity Scale schema (FSS) during a self-treatment course expressed in % from individual start score.

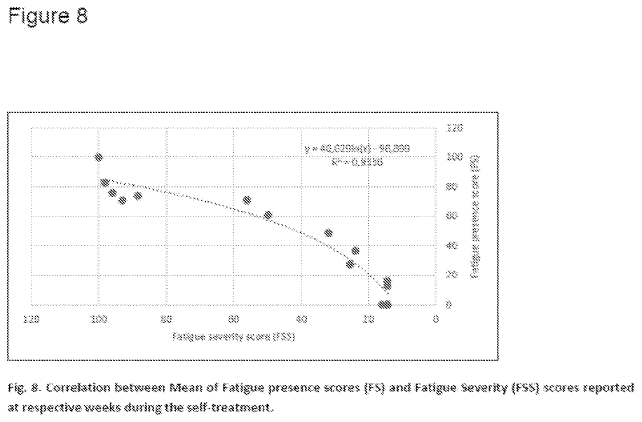

FIG. 8. Correlation between Mean of Fatigue presence scores (FS) and Fatigue Severity (FSS) scores reported at respective weeks during the self-treatment.

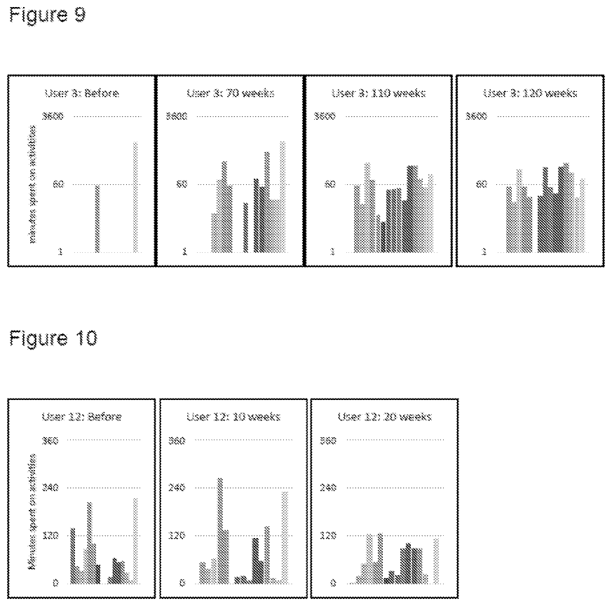

FIG. 9 visualizes the improvement of the HRQoL quality of life through increased DEATH- and SHAFT-activities during the treatment for P3 (user 3).

FIG. 10 visualizes the improvement of the HRQoL quality of life through increased DEATH- and SHAFT-activities during the treatment for P12 (user 12).

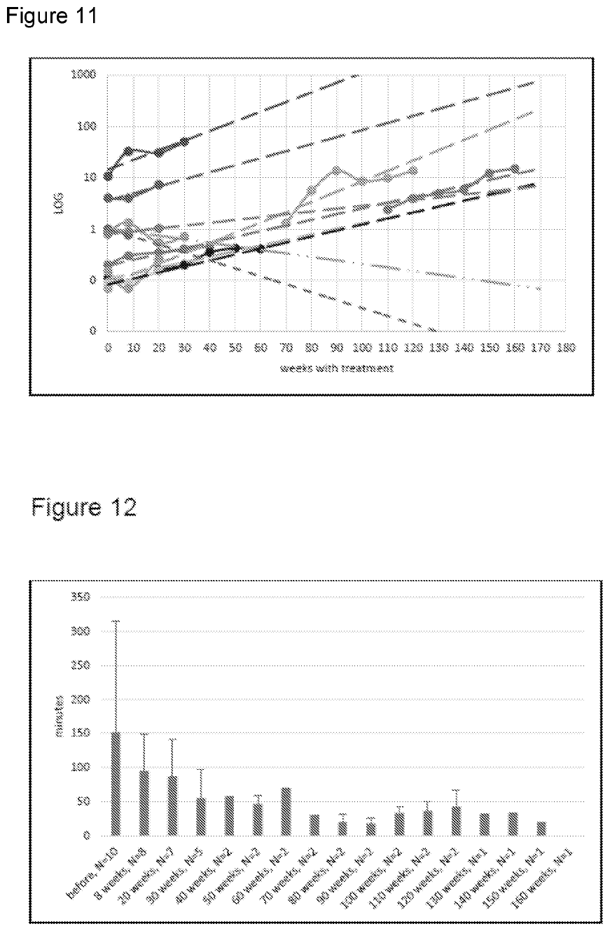

FIG. 11. Visualized improvement of the life quality assessed through The Coefficient of Efficiency of a Daily Life (CEDL), logarithmic scale applied to show the improvement in all users of the treatment.

FIG. 12. Decrease in Mean of Sleep Onset Latency (SOL) during self-treatment course.

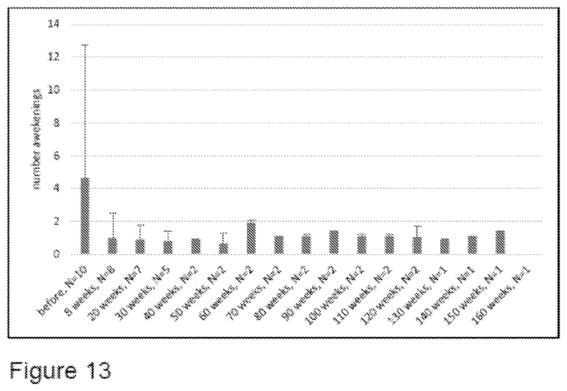

FIG. 13. Decrease in Mean number of awakenings per night during self-treatment course.

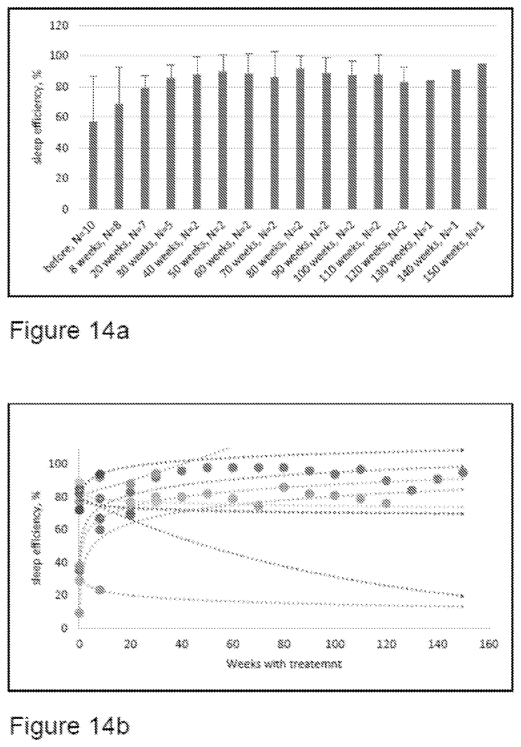

FIG. 14a. Mean of Sleep Efficiency during treatment self-course.

FIG. 14b. Individual scores on Sleep Efficiency at different time points during self-treatment course. Each point corresponding to the mean from 14 nights observations. S.D. are not shown.

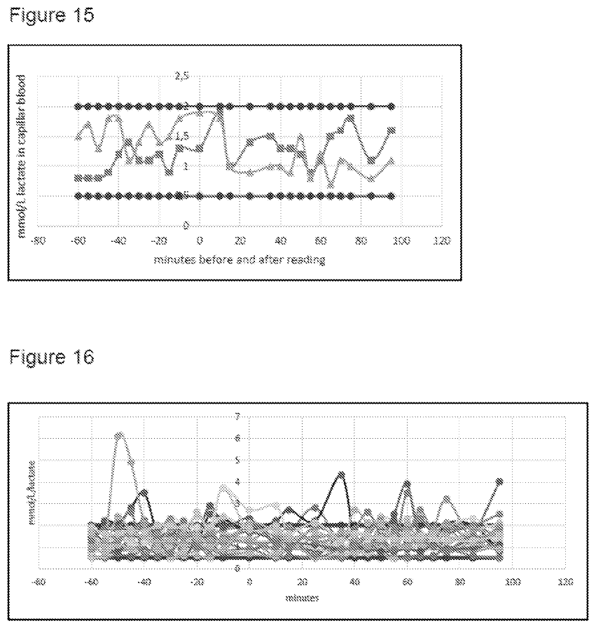

FIG. 15. Typical Lactatogram in healthy persons before, during and after reading of standardized scientific paper in English. Monitoring started 60 minutes before reading (from −60 min to 0 min), during the reading (0-15 min) and after reading (15-95 min). The person A in green squares, and person B in orange triangles.

FIG. 16. Lactatograms of healthy persons, N=20. Normal upper boarder line of 2.0 mmol/L, and normal bottom boarder line of 0.5 mmol/L—both in bold red.

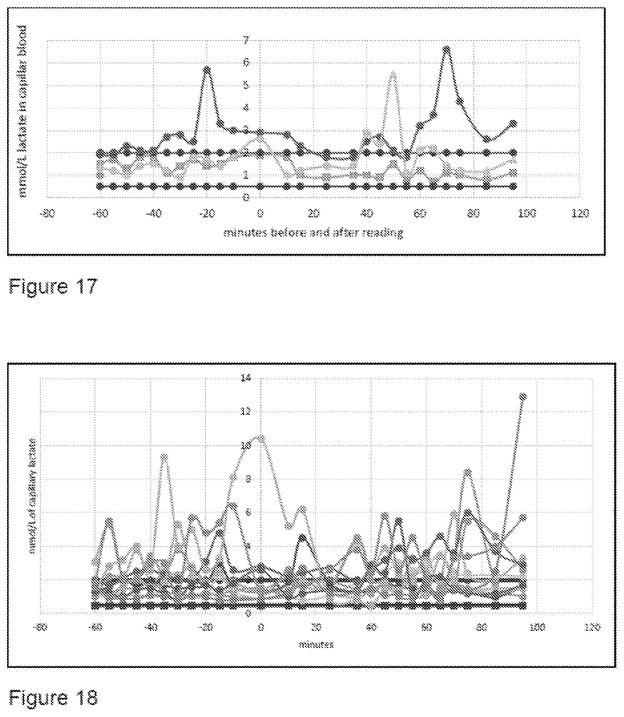

FIG. 17. Comparative picture of Lactatograms from healthy person (orange line with squares) and persons with authorized ME/CFS/SEID (grey triangles and green circles) diagnosis based on Canadian criteria (2003).

FIG. 18. All lactatograms from 10 persons with diagnosis ME/CFS/SEID. The normal range lines are shown in red squares—from 0.5 to 2.0 mmol/L. The maximal measured lactate was 24.7 mmol/L in person 13 (the same as P9 in other examples) is not shown here because of the dominance of the scale.

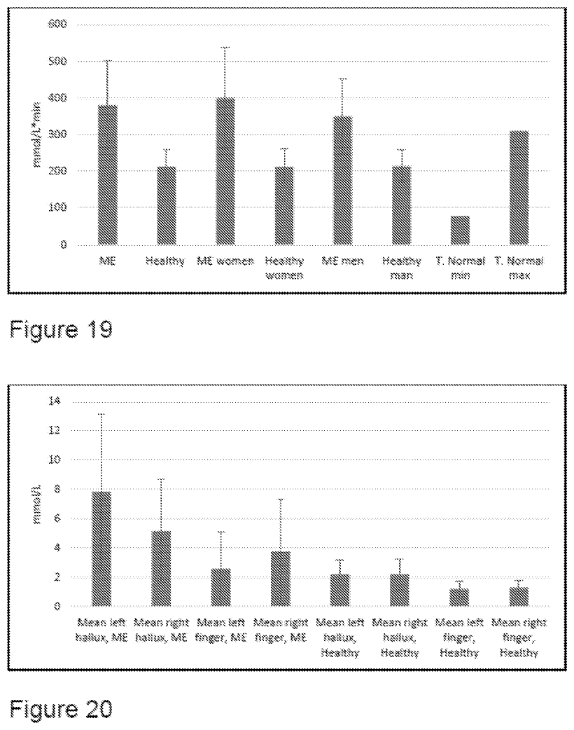

FIG. 19. Mean Total Lactate Load calculated as AUC₁₅₅ in healthy and ME group and their gender under groups. Two columns at right side represents the normal minimal AUC₁₅₅ load, of all measurements of capillary lactate were 0.5 mmol/L or 2.0 mmol/L.

FIG. 20. Mean Lactate values in extremities of healthy volunteers and ME/CFS/SEID affected.

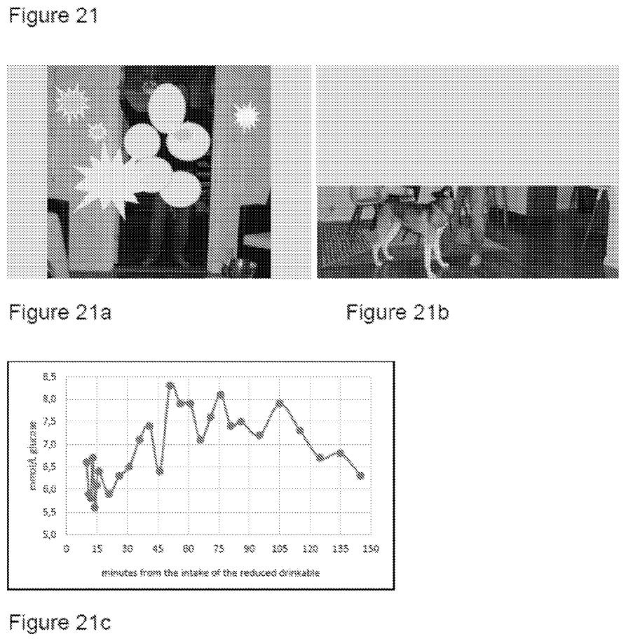

FIG. 21a—field of view disappeared within 3-10 minutes post intake of reduced drinkable; FIG. 21b—field of view disappeared within 10 to 25 minutes post intake.

FIG. 21c—Measured levels of glucose in a capillary blood during 145 min of accident.

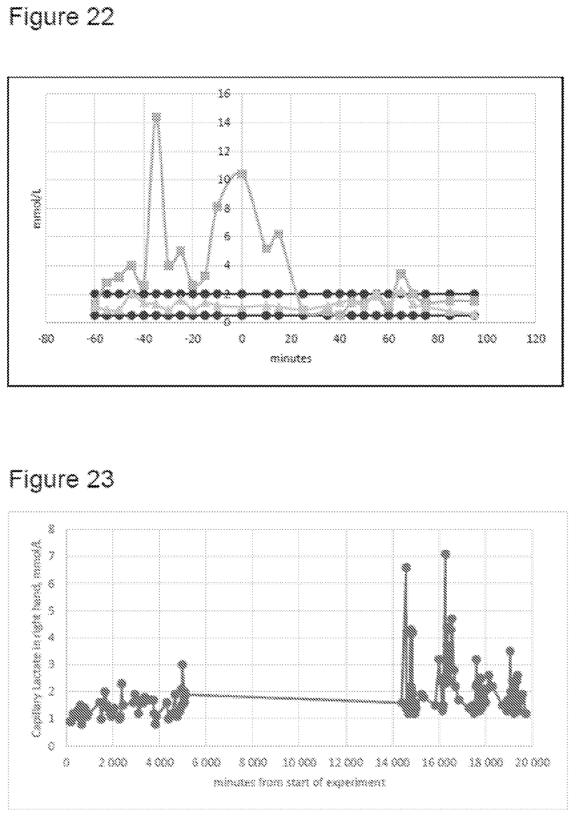

FIG. 22 Lactatogram from person (user) 8, before (grey squares) and 20 weeks with treatment (yellow triangles). 0 minutes corresponds to the start of reading. At 10^{th} minute the person had taken active drinkable for the very first time and profound reduce in capillary lactate could be seen during following 70 minutes.

FIG. 23. Capillary lactate in right hand of P1 for 3 days with treatment (0-3877 min), 1^{st} day without treatment (4319-5097 min), day 6 and day 7 without treatment (14399-16844 min) and day 1 and 2 after re-start of the treatment (17278-18284 min).

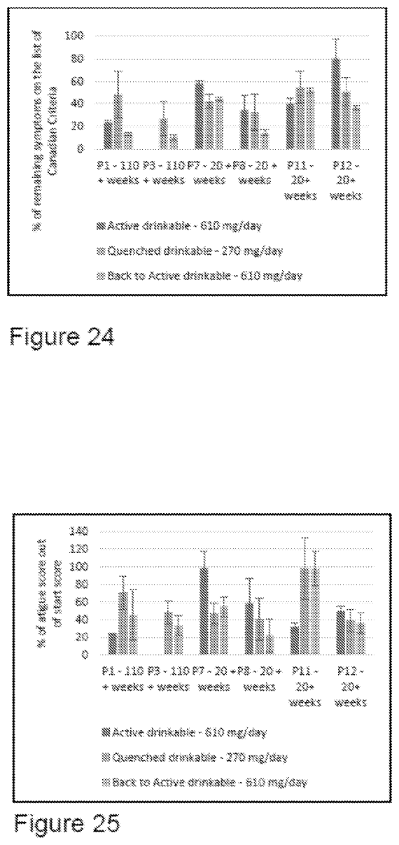

FIG. 24. Placebo-test: symptoms burden, individual data during test course, N=6: Active drinkable in 20-110 weeks before test+3 days; quenched drinkable for 5-22 days and back to active drinkable for 5-22 days: Remaining percent of symptoms was calculated from individual start score before treatment.

FIG. 25. Placebo-test: fatigue presence, individual data during test course, N=6: Active drinkable in 20-110 weeks before test+3 days; quenched drinkable for 5-22 days and back to active drinkable for 5-22 days. Remaining % of fatigue calculated from start score before treatment.

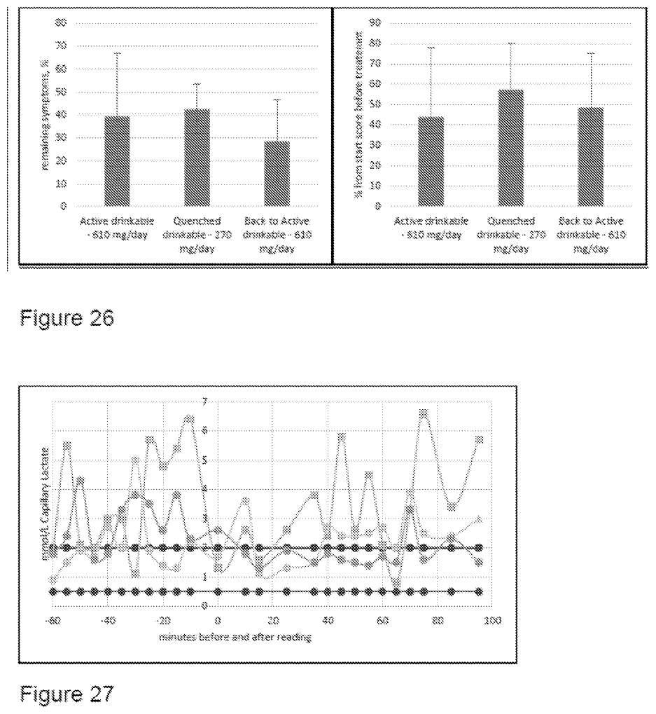

FIG. 26. Placebo-test: A-mean of remaining symptoms and B-mean of remaining fatigue presence, both for N=6: Active drinkable in 20-110 weeks before test+3 days; quenched drinkable for 5-22 days and back to active drinkable for 5-22 days. Remaining percent of symptoms was calculated from individual start score before treatment.

FIG. 27. Lactatogram from person (user) 7, before (grey squares); 20 weeks with treatment (yellow triangles) and after 22 days on quenched drinkable. 0 minutes corresponds to the start of reading. At 10^{th} minute the person had taken active drinkable for the very first time and profound reduce in capillary lactate could be seen during following 70 minutes.

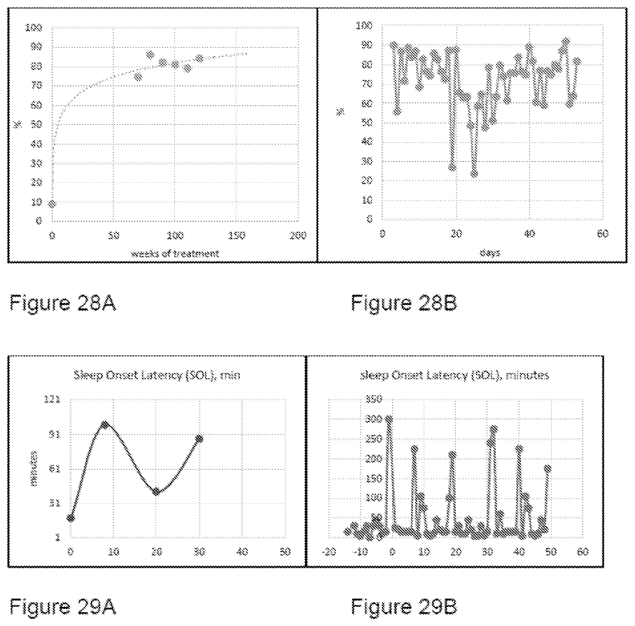

FIG. 28. A—Sleep efficiency improvement during 120 weeks of treatment with active drinkable in P3; B—Night-by-night Sleep efficiency fluctuations during placebo-test schedule in P3.

FIG. 29. A—Sleep Onset Latency improvement during 30 weeks of treatment with active drinkable in P7; FIG. 28B—Night-by-night Sleep Onset Latency fluctuations during placebo-test schedule in P7 (from day 36 P7 was on 20% lowered dose of oxalates in active drinkable).

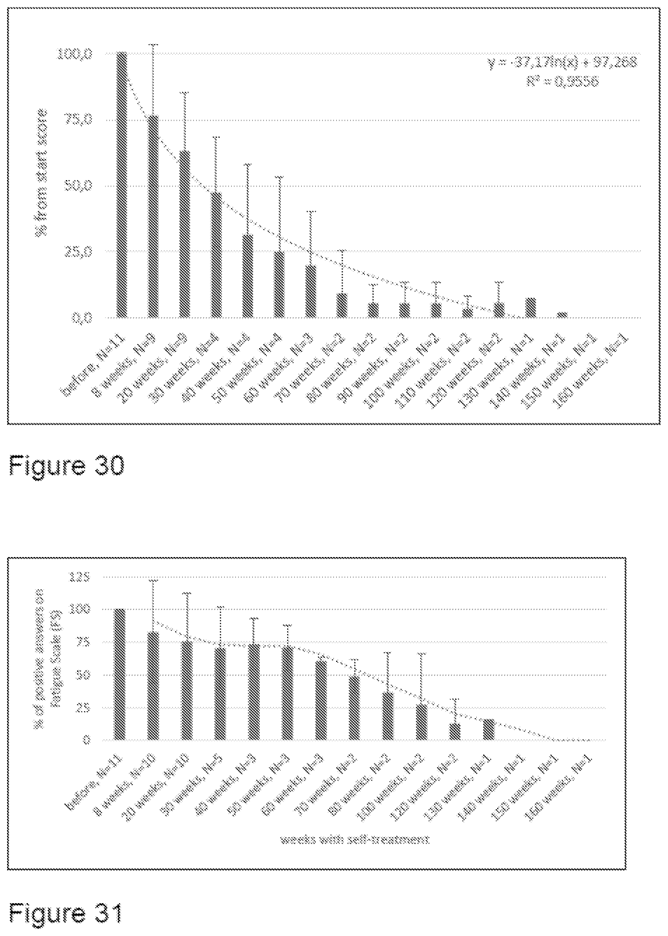

FIG. 30. Effect of treatment of patients with chronic CFS/ME given active drinkable (oxalate) as measured by score on the list of Canadian Criteria as mean of remaining symptoms as % from start.

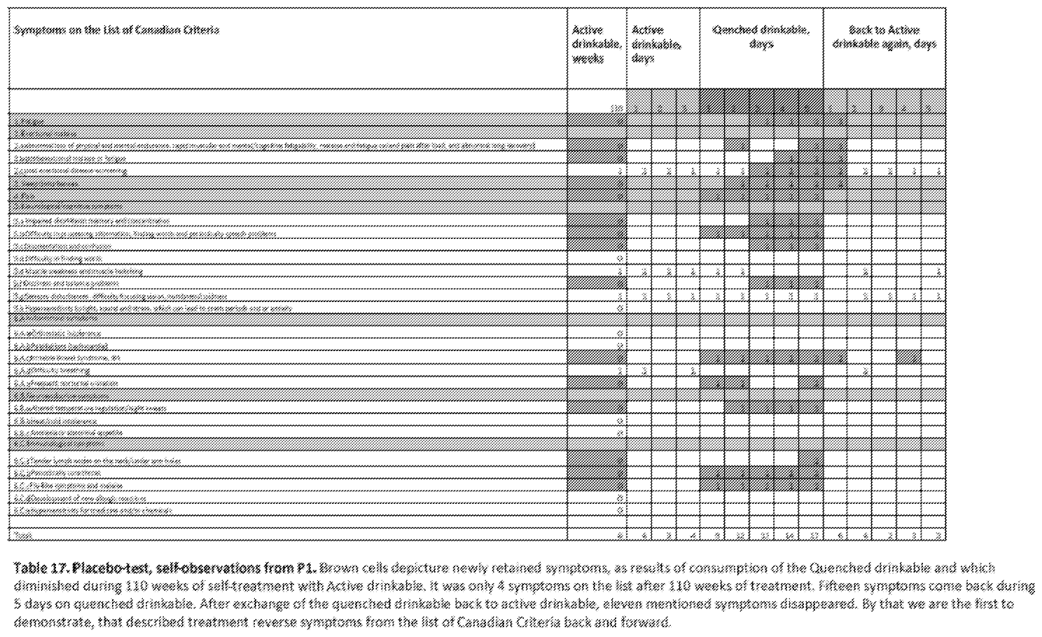

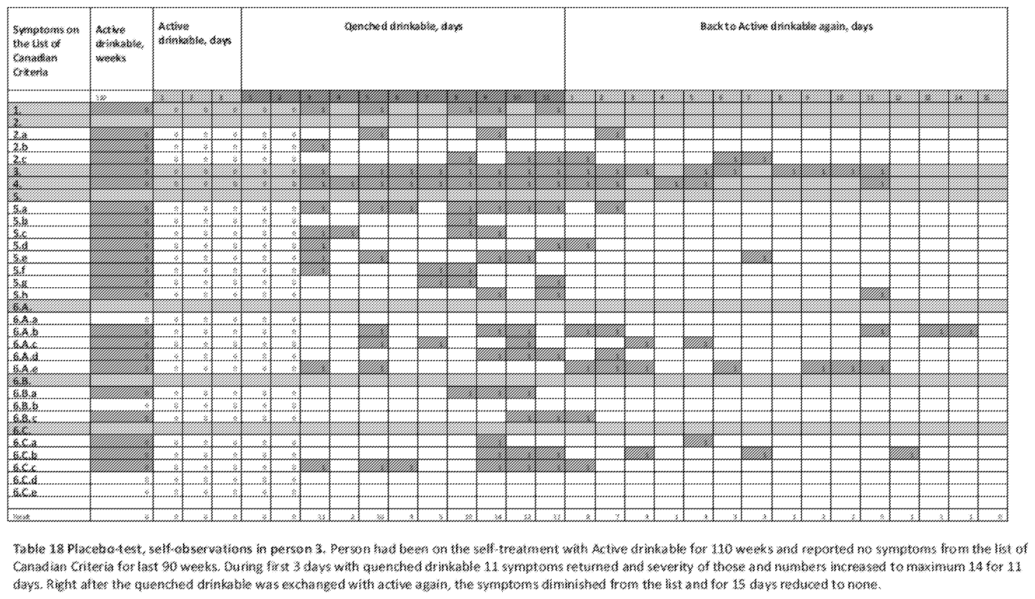

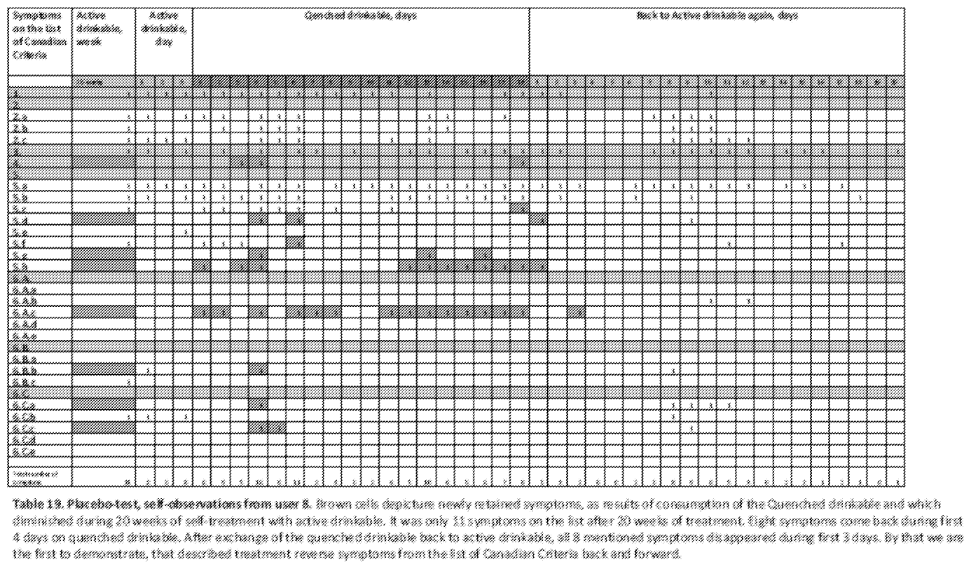

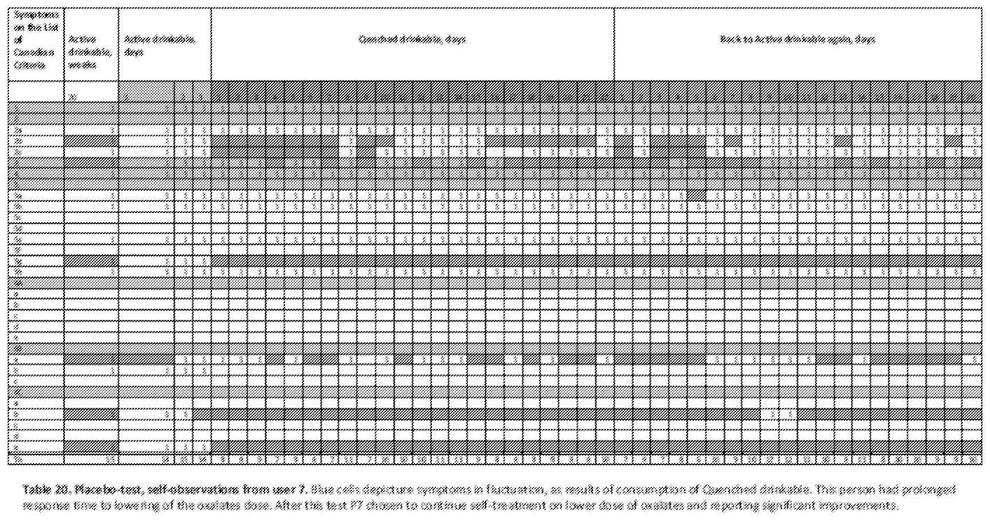

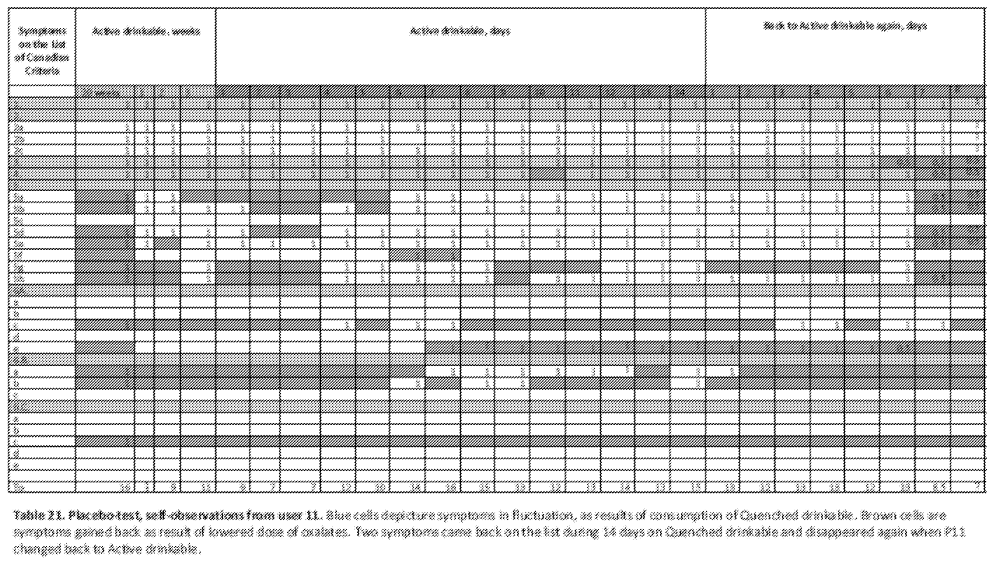

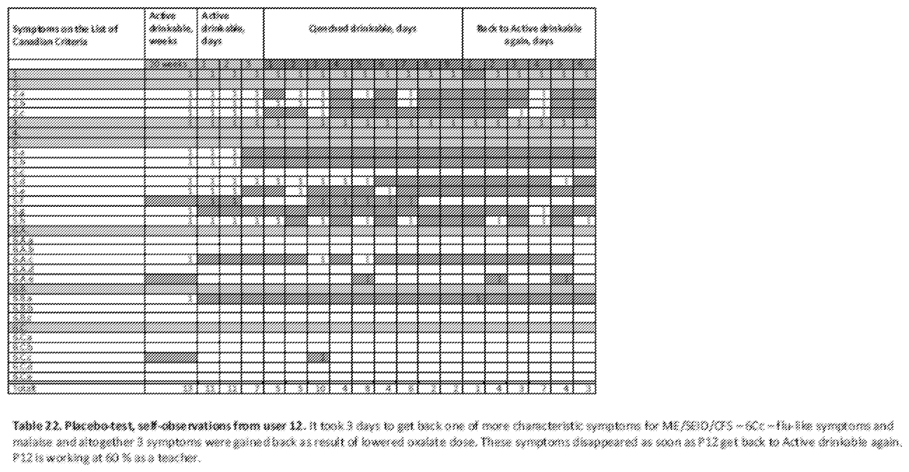

FIG. 31. Effect on CFS as measured by decrease in remaining score on Fatigue Scale schema (FS) in patients given active drinkable (oxalate).

### EXAMPLE 1—ACTIVE COMPOUND

We have shown that compositions containing certain amounts of oxalate or oxalic acid have an effect on CFS/ME/SEID. The active compound of the present invention has been given to the subjects as a component of a drinkable solution, naturally occurring in raw ingredients, not artificial or added. 250 ml drinkable solution were given each day, and the daily dosages of oxalate or oxalic acid were 610-710 mg, due to seasonal variations.

To check the effects of a similar composition with significantly lower concentration of soluble oxalates, we have prepared a “quenched composition”. The term “quenched composition” is used in order to indicate that we actually have lowered concentration of soluble oxalates or oxalic acid in the active composition by chemical transformation of soluble oxalates into insoluble. Insoluble oxalates are crystals, which are insoluble in water and thus, have reduced capacity to be absorbed into a blood stream, and therefore, potentially cannot have any therapeutic effect. The active drinkable solution has been added 0.5 g calcium carbonate (CaCO₃) to one liter. The reaction resulted in formation of insoluble oxalate crystals, which are formed from any type of soluble oxalates present in active drinkable solution as a result of exchange between carbonate group of calcium carbonate to oxalate. By any type soluble oxalates we mean soluble oxalates with solubility in water higher or equal to that for calcium oxalate. When we use the term “oxalates” in a text, we also include all oxalates and oxalic acid. In HPLC system they will give the same peak and could not be distinguished.

Preparation of the “Drinkable” and “Quenched Drinkable”

Active drinkable was prepared by mixing the fruits, greens, vegetables and nuts in proportions necessarily to achieve desirable concentration of soluble oxalates in a final product—active drinkable. Leading levels of oxalates in different raw ingredients could be find in Honow & Hesse (2002). The desirable concentration was determined experimentally in order to have therapeutic effect on symptoms relief in subject 1. For that active drinkable solutions with graded doses of oxalates were consumed at prescribed times during a day: each time a new dose, in increased order. The best dose was tested for additionally couple of weeks to confirm the long-term persisting therapeutic effect. The desirable dose of oxalates was determined as minimal dose taken in one portion drinkable solution which gave therapeutic effect during 5 consecutive hours on pain relief as short term-effect, and for at least for 14 consecutive days as long-term effect, and did not exceed the daily acceptable dose for oxalic acid.

To see if therapeutic effect was associated with concentration of oxalates in a drinkable, a number of recipes with alternative raw ingredients were tested (Table 2). For that raw ingredients were chosen from the published anywhere in literature concentration of oxalates and balancing the recipe in respect to other nutritionals and sugars, so the nutritional values were as much equal as possible for all recipes. The therapeutic effect was tested experimentally on subject 1 as described above.

Quenched drinkable was prepared according to the same recipe as active drinkable solution, when 0.5 g calcium carbonate (CaCO₃) was added and mixed properly. Excessive formation of CO₂ evidenced for the successfully performed exchange between carbonate and oxalate groups and crystal formation of insoluble oxalates. Quenched drinkable was used to evidence the dose-dependence of a therapeutic effect and by that distinguishing the placebo from the real effect. The taste, appearance, fragrance, colour of the quenched and active drinkable solutions were not distinguishable by humans. The crystals were suspected to the FTIR spectra to confirm the structure of the reaction product.

Chemical Composition of Analyses of the “Drinkable” and “Quenched Drinkable”

We describe in details below how we quench the active drinkable in order to obtain the quenched solutions.

In Tab. 1 the results of chemical composition of drinkables are reported as mean value of 10 randomly chosen liters from production batch of 160 liters. The amount of oxalate is not indicated in the table 1 below and discussed later. The results in Tab. 1 show that differences in measured values were not significant, rather due to analytical variation than real differences. It is important to emphasize that amount of sugars and energy value was almost the same in those two drinkable solutions. The only significant differences (marked with *) were documented for the amount of calcium, thus 0.5 g of calcium carbonate (CaCO₃) was added to quench the drinkable (described in detail below); **—for magnesium and ***—for iron. All analysis of nutritionals were performed at German Institute of Food Technology, according to accredited internal and external methods as given in table. Accreditation is valid for methods marked with A.

TABLE 1
Nutritional composition of two drinkable solutions: active and quenched.

| | | | Measured | | |
|---|---|---|---|---|---|
| | | | value in a | Measured | |
| | | | dose of | value in a | |
| | | | 290 g | dose of | |
| | | | quenched | 290 g | |
| | | | drinkable | drinkable | |
| | | | with no | with | |
| | | | therapeutic | therapeutic | |
| Parameter | Calculated as | Method | effect | effect | Units |
| Moisture | | DIL256-1A | 259.84 | 267.96 | g |
| Ash | | DIL259-2A | 1.19 | 1.13 | g |
| Raw Protein | N*6.25 | L17.00-18A | 1.51 | 1.57 | g |
| Total Fat | | DGFC-III 19 | 1.02 | 1.04 | g |
| | | (00)A | | | |
| Carbohydrates | | Calculation | 24.59 | 24.21 | g |
| Fiber | | L00.00-18A | 2.47 | 2.76 | g |
| Sucrose | | DIL789-9A | <0.58 | <0.58 | g |
| Glucose | | DIL789-9A | 8.47 | 8.38 | g |
| Fructose | | DIL789-9A | 16.12 | 15.83 | g |
| Maltose | | DIL789-9A | <0.87 | <0.87 | g |
| Lactose | | DIL789-9A | <0.87 | <0.87 | g |
| Sugar, sum | | Calculation | 24.59 | 24.22 | g |
| Ascorbic acid | | QMP_504_KL_52_020A | 46.98 | 50.17 | mg |
| Thiamine | | DIN EN | 0.06 | <0.03 | mg |
| | | 14122:2010A | | | |
| Riboflavin | | DIN EN | 0.15 | 0.15 | mg |
| | | 14152:2006A | | | |
| Niacin | | USP 34. | 0.75 | 0.81 | mg |
| | | Method 441 | | | |
| Pyridoxine | | DIN EN | 0.23 | 0.23 | mg |
| | | 14663:2006A | | | |
| Folate | | DIN EN | 0.09 | 0.10 | mg |
| | | 14131:2003 | | | |
| Tocopherol | | VO (EG = 152/ | 1.16 | 1.04 | mg |
| | | 2009, IV, B | | | |
| Saturated fat | Triglyceride | DIL770-16A | <0.29 | 0.41 | g |
| Monosaturated fat | Triglyceride | DIL770-16A | <0.29 | <0.29 | g |
| Polysaturated fat | Triglycerid | DIL770-16A | 0.70 | 0.52 | g |
| Sodium | Na | DIL828-8A | <0.06 | <0.06 | g |
| Salt | Na*2.5 | Calculation | <0.58 | <0.58 | g |
| Phosphorous | P | DIL1014-5A | 0.04 | 0.04 | g |
| Potassium | K | DIL938-9A | 443.70 | 452.40 | mg |
| Magnesium | Mg | DIL937-9A | 1.45** | 27.09** | mg |
| Calcium | Ca | DIL931-9A | 1191.90* | 475.60 | mg |
| Iron | Fe | DIL962-6A | 1.07*** | 2.67*** | mg |
| Zink | Zn | ICP-OESA | <0.58 | <0.58 | mg |
| Copper | Cu | DIL940-5A | <0.58 | <0.58 | mg |
| Manganese | Mn | DIL1015-5 | 0.41 | 0.44 | mg |
| Selenium | Se | DIN EN | <0.58 | <0.58 | mg |
| | | 15763, | | | |
| | | mod./F | | | |
| Energy value | | Calculation | 501.70 | 498.8 | kJ |
| Energy value | | Calculation | 118.90 | 118.90 | kcal |
| Cholesterol | | L20.01-13A | <2.9 | 3.48 | mg |

Levels of Soluble and Insoluble Oxalates in Active Drinkable and Quenched Drinkable Solutions

Methodology. To ensure that therapeutic effect was dose-dependent only on oxalic acid and oxalates, also content of other organic acids in drinkable solutions were investigated.

Concentration of oxalates, oxalic acid and other organic acids were measured by HPLC internal standard method at Synthetica A S, Oslo, Norway to assess for content in the different recipes and in active and quenched drinkable. Oxalic acid (Fluka, cat. Nr.75688 lot no 1192734), L-tartaric acid (Aldrich, 25, 138-0), Malonic acid (Fluka, 63301), DL-Malic acid (Fluka, 02308), Citric acid (Merck, 79807-1) were used as external standards. Drinkable solutions were centrifuged at 4000 rpm for 10 min and supernatant collected and centrifuged for the second time. The sample was filtered through a 0.45 m syringe filter followed by second filtration through a 0.2 m syringe filter, and analyzed by HPLC. HPLC conditions were as following: ACE 5 C18 column with 250 mm×4.6 mm i.d. (Part. No. ACE-121-2546) connected to Agilent Infinity 1260 quantifying HPLC system with detector DAD, 10 mm flow cell. Detection at 214 nm at flow rate 0.7 ml/min and with 50 mM phosphoric acid (sodium) buffer solution, pH 2.8 as mobile phase.

Water (VWR, 23595.328, lot. Nr. 17B174005, and HCl (Sigma Aldrich, 30721, STBH0830) were used in preparation of the samples for quantification of soluble oxalates in drinkables. For that 50 ml of each sample was accurately diluted three times and transferred to a 250 ml flask, by using 100 ml of water, HPLC quality. Quantification of soluble oxalates was performed on supernatants of active drinkable and quenched drinkable produced after centrifugation as described above. Calculated difference between those two values gave calculated value for fraction of soluble oxalates transformed into insoluble oxalates by treatment with CaCO₃. Quantification of total oxalates was performed on supernatants of the samples treated with HCl prior to centrifugation, modified from Al-Wahsh et al (2012). For that 50 ml of each sample was accurately diluted three times and transferred to a 250 ml flask, by using 10 ml of 2N HCl. The samples with HCl were further incubated at 80° C. for 30 minutes with vigorously shake from time to time, equal for all samples. Insoluble oxalates were calculated as difference in concentration of soluble from total oxalates. The HPLC conditions were as following: Qualified Instrument Agilent Infinity 1260 with Open Lab Chemstation, ACE 5 C18 column with 250 mm×4.6 mm i.d. (Part. No. ACE-121-2546) at 15° C., injection volume of 3 and gradient of A: 50 mM phosphoric acid (sodium) buffer solution, pH 2.8 and B: 100% CAN as mobile phase. Detection through 10 mm flow cell at 214 nm. Gradient table: start 100% of A; 15 min-100% A; 16 min-5% A and 95 B; 25 min-A and 95% and 26 min-100% A, at 0.7 ml/min flow. The column was washed with CAN for 10 min to ensure elution of potential non-polar compounds in the samples between the runs.

Duplicate standard solutions of oxalic acid were prepared in five different concentration levels for use as external calibration standards: preparation I in the range of 0.06-1.02 mg/ml and preparation II—0.07 to 1.03 mg/ml. The slope (2268.7) from the calibration curve was used for calculation of oxalate in the samples according to formula:

$$C oxalic acid ( \frac{mg}{ml} ) = \frac{Peak area oxalic acid}{Slope reference standard}$$

The same formula and approach was used for quantification of any other organic acid.

Results. Quantification of organic acids, including L-tartaric, Malonic, DL-Malic and Citric acid was performed by comparing the peak areas (FIGS. 1 and 2). Concentration of oxalic acid was most affected by this treatment. This was in compliance with known action of CaCO₃, which first react with the strongest acid in the mix solution—here, oxalic acid. Mean value of measured concentrations of soluble oxalates in 8 randomly chosen untreated with HCl samples of active drinkable was 0.71±0.04 g/L oxalate (or corresponding to oxalic acid anions in HPLC system). This is corresponding to 177.5 mg per dose of 250 ml drinkable. Seasonal variation of soluble oxalate content was documented in active drinkable: 177.5 mg per dose (ingredients are from Italy, November 2017, Tab. 2) and 152.5 mg per dose (ingredients are from Norway and Italy, December 2016, Tab.2).

Concentrations of soluble oxalates were measured in different recipes two times to exclude significant seasonal variation (Tab. 2). The lowest concentration of soluble oxalates was measured in 6-Quenched drinkable, followed by recipe 3-Rhubarb and 1-Strawberry. The highest concentrations were measured in 5-Active drinkable and 4-Banana. Presence of satisfying and long-lasting therapeutic effect was experimentally confirmed only for recipe 5-Active drinkable. Effect from 4-Banana was unpleasant and too tough, while effect from the 3-Rhubarb was unpleasant and effects from both were not lasting long enough either on the short-term of 5 hours or on long-term of 14 days. There was no or reduced effect from the 1-Strawberry drinkable, despite that concentration of soluble oxalates was in the same range as for 5-Active drinkable: 0.69 vs 0.61 mg/L. At least three parameters, which we did not solved in this attempt, could explain the reduced or lack of effect from 1-Strawberry: the ration between soluble and insoluble oxalates and types of soluble oxalates and ratio between them. Quantification of the total oxalates in the solutions after treatment with HCl failed. However, the concentration of remaining in solution soluble oxalates after treatment with CaCO₃ and before treatment could be compared. The calculated ratio soluble:insoluble in 1-Srawberry was 1.5, while in 5-Active drinkable the corresponding ratio was 1.9. This ratio somehow mirrors the composition of types of soluble oxalates in different recipes.

We concluded that to achieve and maintain a pleasant therapeutic effect with satisfying pain relief and long-term effect the concentration of soluble oxalates should be above 0.39 mg/L as in 3-Rhubarb but less than 0.87 mg/L as in 4-Banana. Additionally, ratio between soluble and insoluble oxalates might have influence on the therapeutic effect. Because, therapeutic effect was documented only from 5-Active drinkable and not from 1-Strawberry, we conclude that the ratio between soluble and insoluble oxalates should have influence on the therapeutic effect. Moreover, one important detail was not possible to solve experimentally, but which has significant impact on the therapeutic effect—types of soluble oxalates and ratio between them. It is not possible to distinguish between oxalates by HPLC analysis and therefore this work should be done experimentally on the subject 1 in a future, by testing different combinations of different types of soluble oxalates in a mixture. Development of special analytical tools will also be an option. At this moment, such methodology is not available to us. We conclude that at least four parameters impact the therapeutic effect of a drinkable: concentration of soluble oxalates (1); ratio between soluble and insoluble oxalates (2); types of soluble oxalates (3) and ratio between different types of soluble oxalates (4).

Remaining concentration of soluble oxalates in 6-Quenched drinkable, which is the same as 5-Active drinkable but after the treatment with CaCO₃, was 0.27 mg/L in November 2017 and 0.18 mg/L in December 2016. Thus, confirming that treatment with CaCO₃ in November 2017 gave lowering of the soluble oxalates by 56%. This drinkable was used in placebo-test.

TABLE 2
Oxalate and oxalic acid concentrations in samples of different recipes
tested for presence of therapeutic effect. The first value is from
November 2017 and in parenthesis those are from December 2016.

| | | | | Calculated | |
|---|---|---|---|---|---|
| | | | | Fraction of | |
| | | | | Insoluble | |
| | | | | oxalates | |
| | | | | produced | |
| | | | Remaining | due to | |
| | | | Soluble | treatment | |
| | | | oxalates in | with CaCO₃ | Amount of |
| | Therapeutic | Soluble | quenched | (precipitate | soluble |
| | effect | oxalates in | drinkable, | suspected | oxalates in |
| | (yes/no) of | untreated | g/L after | to FTIR | 250 ml |
| I.D. of | untreated | sample, | Treatment | analysis), | drinkable, |
| drinkable | drinkable | g/L | with CaCO₃ | g/L | mg |
| 1-Strawberry | no | 0.69 (0.92) | 0.24 (n.d.) | 0.45 (n.d.) | 172.5 (230.0) |
| 3-Rhubarb | yes, not | 0.39 (0.16) | 0.12 (n.d.) | 0.27 (n.d.) | 97.5 (40.0) |
| | lasting | | | | |
| 4-Banana | yes, not | 0.87 (1.04) | 0.40 (n.d) | 0.47 (n.d.) | 217.5 (260.0) |
| | lasting, | | | | |
| | unpleasant | | | | |
| | effect | | | | |
| 5-Active* | Yes, long- | 0.61 (0.71) | 0.27 (0.18) | 0.34 (n.d.) | 152.5 (177.5) |
| (Spinach) | lasting | | | | |
| 6-Quenched* | no | Treated with | 0.27 (0.18) | 0.34 (n.d.) | 68.5 (45.0) |
| (Spinach) | | CaCO₃ | | | |

*two drinkable solutions: active drinkable and quenched drinkable.

Analyses of the Spontaneously Formed Precipitate and Crystals Produced During Treatment with CaCO₃

Processing and methodology. Batch of 160 drinkable doses of 250 ml was produced at German Institute of Food Technologies (DIL) with help of High Pressure Processing (HPP) technique. High pressure treatment was carried out in a batch, 55-L-industrial unit (Wave 6000/55, Hiperbaric, Burgos, Spain). Freshly made drinkable was portioned into 250 ml plastic PET bottles and treated at 600 MPa for 3 min holding time. Bottles were kept at 7±1° C. until chemical and microbiological analyses were performed. Bottles were subjected to microbiological examination each second week. Microbial analyses were performed at DIL on total viable count, yeast and molds. In addition, the colour was measured (L*, a* and b* values, Konica Minolta Spectrophotometer CM-600D at 20° C. and standard illuminate light source D65). For cell enumeration, collected samples were serially diluted in Maximum Recovery Diluent CM733 (Oxoid, Deutschland GmbH, Wesel, D.) and surface-plated onto different culture media. After incubation, plates were counted and the results expressed as cfu/ml. Detection limit was 10 cfu/ml. The microbiologic analyses were all accredited.

Test for therapeutic effectivity during self-life of 6 weeks. Each bottle containing 250 ml of drinkable were consumed by a subject 1 accordingly to the treatment schema. The symptoms, stamina and cognitive performance were self-observed and self-reported at least during 8 time points during a day. This experiment take place during week 130 to 133 after start of treatment, and at this time user was in a good physical and cognitive form and reported minor number of symptoms. Each time before consumption of the dose, the bottle was examined in a good light for any deterioration signs.

Analysis of crystalline powder spontaneously formed during the storage. Crystals spontaneously formed during the storage of the HPP-treated drinkable were washed out as described in Da Costa et al (2009) and dried prior analysis. The same washing procedure was performed on crystals formed during the reaction of active drinkable with calcium carbonate (CaCO₃). Analysis were performed at Department of Chemistry at University of Bergen, The Norwegian NMR Platform, Norway. The samples were analyzed at Laboratorium for FTIR spectroscopy and chemometry at University of Bergen. The FTIR spectra of the three samples were compared with a sample of CaC₂O₄ (calsimum oxalate).

Storage experiment of active drinkable used in treatment. Freshly made drinkable of oxalate mix was analyzed as described earlier, in the same HPLC system, and then stored at 4-7° C. for one week before the HPLC quantification was performed again. All analyses were performed by Synthetica A S, Oslo, Norway. Three randomly chosen liters from production of 70 liters had been analyzed. Each sample was stored in 6 aliquots of 50 ml each in a glass bottles with a tight cap: 3 samples were analyzed untreated to attempt content of soluble oxalates and 3 samples were treated with HCl prior to HPLC to attempt the total oxalates in a sample.

Results

The therapeutic effect was tested by subject 1 and was present until day 21, and approximately from day 19 began gradually deteriorates. Less effect was observed and reported as increased pain in muscles and skeleton, chills, strong headache/migraine, frequent nocturnal urination, tender lymph nodes on the neck and under arm holes, sore throat, flu-like symptoms and malaise, sleep disturbances and post exertional malaise. The symptoms appeared suddenly on day 19^{th} and increased in numbers and severity during following days. At day 21 the situation was not acceptable to tolerate and this type of drinkable was exchanged with freshly made. All symptoms disappeared during first 5 hours after intake of the freshly made drinkable.

Approximately after 14 days some light precipitate was seen at the bottom of each bottle (Photo in FIG. 3). The most of precipitated was observed after day 19^{th} and amount of it continued to grow for additionally 30 days until formation of more substance was not observed anymore. Evaluation of the colour difference (ΔE=1,0-2,0) dependent on storage period after HPP treatment showed “subtly seen difference”. Aerobic total count (TPC) was at the level of 10³ cfu/ml after the pressure treatment. The natural occurring yeast and molds concentration was reduced down to the detection limit (<10 cfu/ml). In Table 3 the results of further examinations are collected.

TABLE 3
Microbiology before and after HPP treatment.

| Active drinkable | HPP treatment | | | |
|---|---|---|---|---|
| Storage at 7 ± 1° C., days | 1 | 14 | 28 | 42 |
| pH-value | 4.15 | n.d. | 4.34 | n.d. |
| Aerob mesophile total bacteria count (cfu/ml) | 7.3*10³ | 1.0*10⁴ | 4.6*10³ | 2.2*10³ |
| Yeasts (cfu/ml) | <10 | <10 | <10 | <10 |
| Molds | <10 | <10 | <10 | <10 |

Results of the microbiological examinations confirmed that under storage the drinkable was sterile and no microbiological activity of the bacteria, yeasts or molds could be found. Therefore, the observed precipitated was suspected to have a chemical nature, more specifically—slow rate exchange between calcium and soluble oxalates to form insoluble calcium oxalate.

Crystals collected from the bottles of 5-Active drinkable (Photo in FIG. 3) had been suspected to the FITR analysis, along with crystals formed during treatment with CaCO₃ from 6-Quenched drinkable solution. Both samples, displayed the same features as calcium oxalate, with vibration bands at 1600 cm−1 (C—O stretching), 1315 cm−1 (C—C stretching), 777 cm-1, 660 cm-1, and 511 cm−1. Thus, we conclude that these samples are in fact calcium oxalate.

Thus, conclude from the FTIR spectra (3.3), the main compound of the insoluble powder from reaction with CaCO₃ was calcium oxalate. Consequently, the fraction of soluble oxalates in untreated samples most likely corresponds to all possible oxalates, which are more soluble in water than calcium oxalate. Magnesium oxalate is 149 times more soluble in water than calcium oxalate, and iron (II), ferrous oxalate is 12 times more soluble than calcium oxalate.

Additionally, we have also conducted HPLC analysis of the Active drinkable versus Quenched drinkable used in Placebo-test. Only the first peak with retention time 3.77 min differ in those chromatograms. Overlaid chromatograms confirm the expectation that the peak corresponds to oxalic acid (anion), e.g. oxalates or oxalic acid. Slope for calculation of organic acid content is given in Table 4 and calculated concentration in samples is given in Table 5.

TABLE 4
Slope for calculation of organic acid content

| Reference acid | Retention time | Conc. I (mg/ml) | Conc. II (mg/ml) |
|---|---|---|---|
| Oxalic acid | Approx. 3.77 min. | 1.05 | 1.56 |
| L-Tartaric acid | Approx. 4.44 min. | 1.08 | 1.53 |
| Malonic acid | Approx. 6.61 min. | 0.99 | 1.71 |
| DL-Malic acid | Approx. 6.35 and 13.32 min* | 1.27 | 1.68 |
| Citric acid | Approx. 14.56 min. | 1.96 | 1.46 |

*Two peaks

TABLE 5
Concentration of soluble oxalates in different types of mixes
of drinkable measured for first time, December 2016.

| | | | Calculated |
|---|---|---|---|
| | | | amount |
| | | | of oxalate |
| | | Calculated | anion in |
| | | concentration | 250 ml |
| | Area | of oxalic | portion |
| | Oxalic | acid | of active |
| Sample | acid | (mg/ml) | drinkable mg |
| Sample Ø_F | 602.5 | 0.71 | 178 |
| Sample Ø_U | 150.8 | 0.18 | 45 |
| Sample B | 886.7 | 1.04 | 260 |
| Sample J | 785.1 | 0.92 | 230 |
| Sample R | 138.2 | 0.16 | 40 |

Conclusion. The reaction of active drinkable and 0.5 g CaCO₃ results in an exchange between CO₃²⁻ group on C₂O₄²⁻ group with formation of insoluble precipitate of calcium oxalate (FTIR spectra), simultaneously as level of soluble oxalates in Active drinkable (FIG. 14) is reduced by 56% (November 2017) or 75% (December 2016). Therefore, we conclude that therapeutic effect of naturally oxalates is firstly associated with presence of necessarily amount (dose) of soluble oxalates. Soluble oxalates, which will exchange the C₂O₄²⁻ group with CaCO₃ can be anything with water solubility equal to calcium oxalate and above.

Storage Experiments

Storage experiments were performed on 5-Active drinkable solution at day of production and one week later. Among 3 analysed samples (liters), only one sample demonstrated the expected pattern in oxalate transformation. The reason for that was the huge experimental insecurity and methodological difficulties in transfer and reduction of samples from 1000 ml to 50 ml. Perhaps, insoluble crystals were not evenly distributed and consequently transferred to the storage bottles. However, we succeeded to demonstrate it in one of three samples and with regard that values were produced from separate treated samples, they are in very good agreement with each other. We documented: decrease in soluble oxalates by 9 mg, increase in total oxalates by 7 mg and increase in insoluble oxalates by 16 mg per portion of 250 ml active drinkable solution. It was earlier concluded that dose of soluble oxalates in one portion active drinkable should be more than 97.5 mg (3-Rhubarb) and less than 217.5 mg (4-Banana) and that there were 55 mg less soluble oxalates per portion of 250 ml in 3-Rhubarb than in 5-Active drinkable (Tab. 2). Subject with ME/CFS/SEID takes 250 ml drinkable 4 times a day and consequently, getting 36-64 mg soluble oxalates less per day available when using a drinkable produced for more than one week ago. This confirmed, what we experienced in practice—active drinkable was less effective already after 1 week of storage in home refrigerator.

### EXAMPLE 2—TREATMENT OF CFS/ME/SEID

This example presents the on ongoing self-treatment of 11 persons, which choose to try this treatment by them self in a hope to get improve their severe health condition.

All 11 cases are patients diagnosed with CFS/ME at the Department of Neurology at different University Hospitals in Norway before they started self-treatment. As a routine in Norway, the procedure to exclude other diseases associated with fatigue, such as standard laboratory testing, endocrinology and brain MRIs were performed before the diagnosis was given. Patients performed all routinely investigations in connection to get diagnosis. All findings were normal.

This is a continuously on-going self-treatment without defined in advance termination point, where users started treatment at different times. The users in this example are in the context of this application are indicated as P1, P2, P3, P4, P5, P6, P7, P8, P9, P11 and P12. Duration of the treatment on Nov. 6, 2017 was 160 weeks (3 years) for P1, 120 weeks for P3, 60 weeks for P4, 50—for P2, 30 for P7 and P8, 20 weeks for P5, P6, P11, P12 and 10 weeks for P9. Fourteen days self-observations were performed before the start and later on at each 10^{th} week. The schema and questionnaire used in observations were the same as used at ME rehabilitation center at Haukeland University Hospital and described in details below.

All persons are responsible for their own treatment, none pre-selection was made. Only exclusions criteria applied were the medicinal drug abuse (Appendix 1). All painkillers, nutritional supplements were excluded from use to minimize the potential cocktail effect.

Self-treatment was introduced according to schema. The necessarily controls, including positive response on first dose, negative and placebo, were performed in the same user at time points as shown in Tab. 4 and described below. Excluded persons are described in Appendix 1.

Positive control. Effect of the very first dose of each part of the treatment were monitored separately. Any changes in symptoms were noted and experienced changes were discussed with the patient. The meaning of this test was to identify potential none-responders or slow-responders. Independently of the results, the users started their treatment from the first dose and continued as described above.

Negative control. This is a report on the self-treatment and do not have any control for placebo in means of a control group. This decision can only be justified by the severe health condition of each user, which was in a negative trend (steadily worsening) before start. On another hand, treatment doses had profound painkiller effect lasting for up to 5 hours and therefore each dose taken or not taken had functioned as a positive control (when taken) and negative control (when not taken) in the same user. Moreover, each of the users had experienced at least one 14-90 days period without a treatment, which resulted in clearly communicated worsening of the condition.

Exclusion of the placebo effect. Two identical formulations of drinkable solutions (Tab. 1 and 2), except for the presence of active substances, were used to discriminate between real effect and placebo: one drinkable called active drinkable containing the active compounds (soluble and insoluble oxalates), while another one—quenched drinkable—was prepared with fade out soluble oxalates, i.e. where concentration of soluble oxalates was reduced by adding a quenching agent.

Each of 11 users was at a different state of recovery and used the treatment for different numbers of weeks when received a supply of quenched drinkable for one week usage with explanation that this was an alternative formulation: P1 and P3 after 110 weeks and the rest of the group after 20 weeks on treatment with Active drinkable. They were asked to use two schema—Fatigue Scale (FS) and Symptoms list (as in § 2) to monitor the eventually improvements of their condition. As, in fact, no improvement were expected, but worsening, the new supply of the active drinkable were given when worsening was communicated by user. The monitoring of the health condition was performed for 3 consecutive days before consumption of the quenched drinkable (start) and until user communicated worsening of symptoms and scope (test) and again until those symptoms disappeared when quenched drinkable was replaced with active drinkable (revers). The time to achieve worsening in symptoms and their scope and consequently, the revers time to achieve the diminishing of the symptoms and scope were noted. It is necessarily to emphasize, that only drinkable solutions were exchanged, not vitamins and alpha-lipoic acid or sugar content or composition. Those were taken as usually and accordingly to the prescribed regiment.

The official diagnosis of ME/CFS is given when Canadian Criteria are fulfilled and other causes to fatigue and malaise are excluded by extensive investigation by specialists. The List contains following symptoms (in parenthesis the symptoms which were monitored in this report):
1. Fatigue (1);
2. Exertional malaise;
   a. abnormal loss of physical and mental endurance, rapid muscular and mental/cognitive fatigability, malaise and fatigue or/and pain after load, and abnormal long recovery) (2);
   b. post exertional malaise or fatigue (3);
   c. post exertional disease worsening (4);
3. Sleep disturbances (5);
4. Pain (6);
5. Neurological/cognitive symptoms
   a. Impaired short-term memory and concentration (7);
   b. Difficulty in processing information, finding words and periodically speech problems (8);
   c. Disorientation and confusion (9);
   d. Difficulty in finding words (10);
   e. Muscle weakness and muscle twitching (11);
   f. Dizziness and balance problems (12);
   g. Sensory disturbances, difficulty focusing vision, numbness/coldness (13);
   h. Hypersensitivity to light, sound and stress, which can lead to crash periods and or anxiety (14);
6. Symptoms (autonomous, neuroendocrine and immunological)
   A. Autonomous symptoms
      a. Orthostatic intolerance (15);
      b. Palpitations (tachycardia) (16);
      c. Irritable Bowel Syndrome, IBS (17);
      d. Difficulty breathing (18);
      e. Frequent nocturnal urination (19);
   B. Neuroendocrine symptoms
      a. Altered temperature regulation/night sweats (20);
      b. Heat/cold intolerance (21);
      c. Anorexia or abnormal appetite (22);
   C. Immunological symptoms
      a. Tender lymph nodes on the neck/under arm holes (23);
      b. Periodically sore throat (24);
      c. Flu-like symptoms and malaise (25);
      d. Development of new allergic reactions (26);
      e. Hypersensitivity for medicine and/or chemicals (27);
7. Symptom stream
   a. Conditional lasted for at least 6 months;
   b. Fatigue started with well-defined debut within weeks;
   c. Fatigue developed gradually;
   d. Fatigue and symptoms are stable or improving;
   e. Fatigue and symptoms are gradually worsened

Monitoring of the symptoms was first performed 14 days prior the start and then 8 weeks after the start and further each 10^{th} week, for 14 consecutive days each time. Thus ME/CFS/SEID symptoms are known to fluctuate thought a day, different parts of the body and vary in intensity, which all makes it difficult to follow, the persons were asked to scan their body for any symptoms at least 7 times a day and at the same times as they took the medication. All symptoms were noted on the daily schema, which were used at the end of the period to recall all ailments. Until week 20 the dosage was ever increased accordingly to the treatment schema and after week 20 all individuals were using the same dosage and regiment.

Number of positive symptoms was different for each person before the treatment and associated with the severity of the health condition. Five persons had full score of 27 out of 27, two persons reported 22 of 27, three persons set 21 of 27 and one person registered 10 of 27 answers.

It is interesting to give a description of the experience with symptom relief in response to a very first dose of drinkable. The common futures on the very first dose of drinkable (250 ml) was re-established body temperature: those who were freezing get warm and vice versa. The pain was reduced all over the body, including headache (§ 2) and all reported that they are feeling relief, energized and easier in the body, while dependents observed them to be significantly more communicative and snuggle in the brain, restored clarity of the speech, more voice strength, lower heart rate, diminishing of jitter in the entire body. The effect was obvious to the present relatives and for the user self. The time it taken to feel the mentioned effect of drinkable was in the range 35-105 min with mean of 60.

All users reported immediately, but not yet persistent, improvement of their symptoms from the very first dose of the treatment had been taken. The relief in their symptoms occurred each time after the dose had been taken and depending on the severity of the health condition lasted for up to 4.5 hours post consumption. Weakest persons experienced that the relief did not last for all 4.5 hours, but at the beginning only for 2-3 hours. With time, this relief-time extended to 4.5 hours in all users.

Another observation all users were agreed on, is that along treatment the symptoms appears less frequently and are much milder.

As shown in Tab. 6 all users reported relief in their symptoms and number of remaining symptoms after first 8 weeks of treatment was in the range of 22 to 10 symptoms left, which corresponds to minimum 22 to maximum 55% relief, with a mean of 25% for 9 users. Some of users reported also that some of the symptoms became weaker and came rarer, but as long as they were commented on the list, they were counted as they were present.

TABLE 6
Remaining number of the positive answers on the List of Canadian
Criteria reported during course of the self-treatment.

| | Weeks of | | | | | | | | | | | | | |
|---|---|---|---|---|---|---|---|---|---|---|---|---|---|---|
| | self- | Number of positive answers from the list of 27 possible | | | | | | | | | | | | |
| | treatment | P1 | P2 | P3 | P4 | P5 | P7 | P8 | P9 | P10 | P11 | P12 | mean | S.D. |
| 1th | 0 | 27 | 27 | 27 | 27 | 27 | 21 | 21 | 20 | 22 | 22 | 10 | 23 | 5 |
| year | 8 | 17 | | | 21 | 17 | 17 | 12 | 22 | 15 | 10 | 14 | 17 | 3 |
| | 20 | 12 | 10 | 12 | 20 | 17 | 15 | 11 | | | 16 | 11 | 14 | 3 |
| | 30 | 5 | | | 19 | | 11 | 10 | | | | | 11 | 6 |
| | 40 | 3 | 12 | 2 | 17 | | | | | | | | 9 | 7 |
| | 50 | 3 | 5 | 1 | 18 | | | | | | | | 7 | 8 |
| 2nd | 60 | 2 | | 1 | 13 | | | | | | | | 5 | 7 |
| year | 70 | 2 | | 3 | | | | | | | | | 3 | 1 |
| | 80 | 3 | | 0 | | | | | | | | | 2 | 2 |
| | 90 | 3 | | 0 | | | | | | | | | 2 | 2 |
| | 100 | 3 | | 0 | | | | | | | | | 2 | 2 |
| 3rd | 110 | 2 | | 0 | | | | | | | | | 1 | 1 |
| year | 120 | 2 | | 0 | | | | | | | | | 1 | 1 |
| | 130 | 2 | | | | | | | | | | | 2 | 0 |
| | 140 | 2 | | | | | | | | | | | 2 | 0 |
| | 150 | 0 | | | | | | | | | | | 0 | 0 |

FIG. 4 demonstrates gradually decrease in symptoms burden by showing the mean of remaining % of symptoms reported. Total number of positive answers on the list was 27, corresponding to the 100% of symptoms burden, however the initial start score was taken as 100% to calculate the outcome shown on FIG. 2.1. Reduction of >50% occurred between 20 and 30 weeks of self-treatment in 9 persons and that only 25% of symptoms are remaining after 50 weeks of self-treatment applied by 4 persons, so far. Three persons used this self-treatment for 60 weeks and achieved reduction below 20%. Two persons self-treated for 120 weeks with mean of 4% of remaining symptoms, where of one of them reported no symptoms from week 80 for 50 consecutive weeks (P3). One person (P1) used this treatment for 150 weeks and reported two symptoms left from week 110 for 30 consecutive weeks and no symptoms left after that.

In conclusion, this treatment with active drinkable (oxalates) provides significant symptom relief from very first dose and during first year of treatment. The effect has been demonstrated to be lasting and persistent for the complete all test period of three and a half years. Subjective experience communicated by users was that feeling to be even stronger and healthier as before the ME/CFS/SEID onset and receiving the diagnosis. They also telling that they have feeling that they are still recovering and they are very excited to see how much healthier them might became.

Pain Reduction

As it is known, the List of Canadian Criteria does support only common and most widespread features and symptoms. However, the symptoms burdens in CFS/ME/SEID patients are not limited to The List. The visualization of the bodily ailments is often used in communication because many patients has impaired cognitive functioning, difficulties to process complex information, find the words and keep concentrated, along with impaired short-, and working memories and even absence of the voice or snowy speech. All that will make communication of the health situation almost impossible. We used the visualization charts, where the users were asked to draw all kind of ailments associated with pain, searing, sting and heat with a red marker and the chills, freezing and cold with a blue. The drawings were assembled on the base of 14-days self-observations undertaken each 10^{th} week.

Pain is the most common and invaliding ailment in CFS/ME/SEID patients. There are many types of pain and they all will be located differently in the body. The most unbearable pain is in skeleton, through all bones, even finger phalanges and collarbones. This pain could be compared to influenza pain in the body, but had lower really unpleasant frequency and remains persistent despite of use of medicinal opiates. Pain in muscles is also flu-like and also similar to muscular pain and trigger points at fibromyalgia. Some may experience severe pain in extremities: in lower extremities, it is often extraordinary feeling of the burning from frost, while pain in upper extremities give association to be quartered by the horse force (in the old fashion). Headache. We had counted at least 8 different locations and types of pain in the head: most common is press feeling as if scull was too small for the brain (1), swim hat (2), pulsation in the fountain (3), back pain (4), facial pain (5); nasal pain (6), iron ring around the forehead (7) and one-sided migraine pain, often combined with pain behind the eyeball (8). Sometimes all pain symptoms are more profound on one side of the body (FIG. 4. panel A, B, F).

As shown in FIG. 5, nine of eleven users communicate reduction of pain along with the treatment.

Decrease in Fatigue Presence (FS) and Severity (FSS)

Fatigue at CFS/ME/SEID means exceptional impotence in any physical or cognitive activity without any understandable/detectable cause. It feels like all body collapsing and one more stimuli will be enough to stop the life. Fatigue is persistent and has to be present at least for last 6 consecutive months before diagnosis is given. Fatigue does not improve by night sleep or day rest. Fatigue is not the same as to be exhausted or tired, in a popular sense of words. Fatigue could be described as absence of any stamina, strength or operating force in whole body at the same time, it is often associated with an unpleasant jitter in whole body and body surface. This jitter is not visible.

However, despite of the low sensitivity of the questionnaires the effect of this therapy could still be documented. The long-term improvement is reported here. Individual start scores were different, therefore all scores reported through self-observations during 14 consecutive days and during treatment course were transformed to % from that individual start score and further statistics were done on % values from each individual. Values given in the figures are the mean of all individual scores at given time, which means very large range of recovery between the subjects. FIG. 6 shows that the Fatigue presence (as measured by the score on FS schema) decreases during the treatment period. The individual progress in Fatigue presence shown in Table 7 below.

TABLE 7
Individual scores on Fatigue Scale (number of points summarized
and equally weighted) schema during the self-treatment course.

| weeks | P1 | P3 | P4 | P5 | P6 | P7 | P8 | P9 | P11 | P12 |
|---|---|---|---|---|---|---|---|---|---|---|
| 0 | 31 | 33 | 33 | 33 | 33 | 26 | 26 | 27 | 29 | 24 |
| 8 | 30 | 33 | 33 | 32 | 33 | 4 | 26 | 32 | 8 | 30 |
| 20 | 29 | 30 | 33 | 24 | 32.5* | 28 | 23 | | 9 | 11 |
| 30 | 28 | 27 | 32 | | | 17 | 5 | | | |
| 40 | 24 | 24 | 30 | | | | | | | |
| 50 | 20 | 19 | 30 | | | | | | | |
| 60 | 19 | 19 | 21 | | | | | | | |
| 70 | 18 | 13 | | | | | | | | |
| 80 | 18 | 5 | | | | | | | | |
| 90 | 17 | 3 | | | | | | | | |
| 100 | 17 | 0 | | | | | | | | |
| 110 | 8 | 0 | | | | | | | | |
| 120 | 6 | 0 | | | | | | | | |
| 130 | 5 | | | | | | | | | |
| 140 | 0 | | | | | | | | | |
| 150 | 0 | | | | | | | | | |

*the half point came from the answers expressed by two numbers, it occurs when fatigue perception are very varying from day to day, and often associated with occurring decrease in fatigue presence.

Decrease in Fatigue Severity

Fatigue severity was monitored by answering the questionnaire (FSS) at the end of the 14 days self-observation period, each 10^{th} week. The raw individually data were transformed to % value in the same manner as for Fatigue Presence data. FIG. 7 give a graphical presentation of the gradual decrease in fatigue severity. Reduction by 50% was achieved between week 40 and 50, and reduction by 75% between week 70 and 80. From week 120 user 1 reported minimal possible score of 9 out of 63. Thus, minimum score in this questionnaire is 1 for each question, it is not possible to get lower score than 9.

User P3 reported lowest score of 9 out of 63 since week 90. This achievement was stable for consecutive 60 weeks in P1 and 50 in P3. Thus it was large interindividual variety due to differences in a condition severity before the treatment, the individual progress is also shown in Table 8 below.

TABLE 8
Individual scores on Fatigue Severity Scale schema
during self-treatment course. The number of
points were equally weighted and summarized.

| Weeks | P1 | P3 | P4 | P5 | P6 | P7 | P8 | P9 | P11 | P12 |
|---|---|---|---|---|---|---|---|---|---|---|
| 0 | 63 | 63 | 63 | 63 | 63 | 63 | 60 | 63 | 60 | 62 |
| 8 | 60 | 63 | 63 | 63 | 63 | 63 | 55 | 63 | 58 | 63 |
| 20 | 57 | 60 | 63 | 63 | 63 | 63 | 52 | | 59 | 54 |
| 30 | 54 | 57 | 63 | | | 63 | 53 | | | |
| 40 | 50 | 54 | 63 | | | | | | | |
| 50 | 24 | 19 | 63 | | | | | | | |
| 60 | 20 | 15 | 59 | | | | | | | |
| 70 | 23 | 17 | | | | | | | | |
| 80 | 19 | 11 | | | | | | | | |
| 90 | 24 | 9 | | | | | | | | |
| 100 | 23 | 9 | | | | | | | | |
| 110 | 21 | 9 | | | | | | | | |
| 120 | 9 | 9 | | | | | | | | |
| 130 | 9 | | | | | | | | | |
| 140 | 10 | | | | | | | | | |
| 150 | 9 | | | | | | | | | |

FIG. 8 demonstrates that data derived from two independent questionnaires—Fatigue Scale (FS) and Fatigue Severity Scale (FSS) are in compliance with each other. FIG. 8 shows the Correlation between Mean of Fatigue presence scores (FS) and Fatigue Severity (FSS) scores reported at respective weeks during the self-treatment. It could serve as a control for compliance of the observations made.

Increased Daily Activity and Improved Health Related Quality of Life (HRQoL)

Health related Quality of life is an assessment of how individual's well-being may be affected over time by a disease, disability, or disorder.

HRQoL is tightly associated with the Activities of daily living which give a meaningful life and private independence. However, because health issues can disturb most basic functioning, for example satisfied breathing, quality of sleep, eliminating wastes (urination, defecation and sweating), feeding oneself, dressing, the Activities of daily living can be also affected. To understand the grade of disability/quality of life, the daily activities could be distinguished into two groups: Activities of daily living (DEATH) and Instrumental

Activities of daily living (SHAFT). The severity of the health condition and lower life quality will be mirrored in a shorter list of first SHAFT- and then DEATH-activities. We addressed these issues through the 14 days self-reports of the daily activities by asking the users to note number of minutes they spent on different activities:

| Activities of Daily | Instrumental of Daily |
|---|---|
| Living (DEATH) | Living (SHAFT) |
| Dressing/bathing/showering | Shopping for groceries and necessities |
| Eating | Cleaning and maintaining the house |
| | (Housework) |
| Ambulating/walking | Managing money (Accounting) |
| Toileting | Preparing the meals (Food |
| | preparations/meds) |
| Hygiene | Telephone/moving within community |

While the ADLs were measured as they are listed in Table above, the IADLs were slightly different organised into a bigger group: “shopping”, “managing money”, “preparing the meals” were all assigned in a group of “self-service”. The complete list of monitored activities with bars colours are

FIG. 9-10 visualizes the improvement of the HRQoL quality of life through increased DEATH- and SHAFT-activities during the treatment.

Severely Invalidated, P3. He was accredited at the community nursing home. The only activity left, before the treatment started was communication with medical personal (green bar) and resting (yellow bar) in FIG. 9. Person was fed through nasal probe and could not turn himself in the bed. From the list of Activities of Daily Living none of activities were remained in use. The nasal probe was removed 7 days after treatment start, user gained energy to chow and swallow the food. During first couple of weeks he also started to be wear of hygiene and started to brush teeth and tolerated to be washed with a cloth. Ability to speak in full voice came around week 40 and eye mask was removed at week 36. Since he showed great improvement: exercising at least 3 times a week at centre for at least 60 minutes each time; using communal transportation back and forward to the centre for 30-120 minutes. On the list of IADLs he operates with accounting, telephone/transportation, preparing the meals, shopping, cleaning without the assistance and exercising in mathematics for 1-4 hours a day.

Moderate invalidated person, P12. None-responder to Rituximab. This person had been recognised as non-responder to Rituximab therapy, altogether 8 doses given during clinical studies at Haukeland University Hospital by Fluge and Mella. The person was in 60% position with reading, writing and social exposure as a part of the job. The graphs are in linear scale. The main achievements during 20 weeks of treatment were increased stamina, which allowed to performed daily activities for longer time, halved (½) need for rest and sleep during day time, increased ability to read, write, listen to music and watch TV. Hiking, driving a car, physical exercises and hobby in house and outside became part of persons daily life on more regular basis and for double duration of time spent on each activity. The results are visualized in FIG. 10.

Increased Daily Activity (Coefficient of Activity or CEDL)

During their recovery, all users did experience that increase activities did not demanded increased rest. To document that, the resting time in bed or daybed and time lying in a bed or daybed while writing, reading or doing other activities, which healthy people will normally do in a sitting position in a chair or sofa, we monitored during those 14-days self-observations. The number of active minutes used to perform DEATH- or SHAFT-activities were divided by number of minutes users have spent in a lying position. We called this value for the Coefficient of Efficiency of a Daily Life (CEDL). The positive trend of the increased Efficiency of a Daily Life for 9 of 11 users shown on FIG. 11.

CEDL coefficient had increased in 8 of 10 persons during their respective treatment durations: P1 by 120 times for 150 weeks; P3 by 200 times for 120 weeks; P4 by 3.7 times for 60 weeks; P5 by 1.7 times and P6 by 1.6 times for 20 weeks both; P8 by 5 times for 30 weeks; P12 by 1.8 times and P11 by 1.3 times for 20 weeks, respectively. In two of ten persons CEDL coefficient get decreased because they are not receiving a full dose of treatment yet: P9 at week 8. In person 7 the CEDL coefficient decreased because of rejection of walking-chair.

Improved Sleep Quality

Poor sleep quality is one of the most troublesome symptoms for ME/CFS affected. The sleep pattern extremely departs from the sleep pattern in healthy persons. The main features are: extremely prolonged time for sleep onset (SOL), extended time in bed (TIB), combined with shorten sleep time (TST), increased number of awakenings after the sleep onset (NWAK) and increased duration of time without the sleep after awakenings (WASO) with extremely poor sleep efficiency (% of time in bed utilized for sleep). The night rest is also disturbed by muscular/skeletal pain, headache and frequent urination. Patients are also reporting no refreshing after night rest. Here are reported values based on notes for 14 consecutive nights at given time points (weeks of treatment).

Sleep Onset Latency

One of the most unpleasant and exhausting symptom along with the pain was extremely prolonged sleep onset latency (SOL). One person could not sleep at night at all (P6) and was not measured for this parameter. P2 had used sleep medicine and have been excluded when it was discovered (Appendix 1), therefore no parameters were calculated for this person. Four out of ten persons had SOL in the range of 159 to 463 minutes, while 3 of 10 persons had SOL in the range of 46-67 minutes. Two of ten persons had almost normal SOL in the range of 18-29 min before start of the self-treatment. Four of 10 persons used help of pharmaceutical drugs for this challenge (P4-6, P11). All of them stopped medication with that just before the start of the treatment.

Most dramatically improvement of the SOL was documented in five of ten persons, those who had more severe disturbances of the sleep pattern, those four persons with longest SOL and P8. Improvement of the SOL had been halved during first 20 weeks in P1, P3 (personal communication), P4 and P5. In the case of P5 the SOL became equal to this measured with use of sleep medication before treatment. The improvement of this parameter characterized as one of the most pleasant and highest desired among users. During 50 weeks P1 experienced reduction of SOL from 241 to 38 minutes, P4—from 300 to 55 min, P3—from 463 to 30 min for 70 weeks and P8—from 29 to 7 minutes. Improvement is stable through 2^{nd} and 3^{rd} year in two persons who get so far.

The decrease in Mean of Sleep Onset Latency (SOL) during self-treatment course is given in FIG. 12 and table 9.

TABLE 9
Sleep Onset Latency, mean of 14 consecutive
nights (Relative Standard Deviation, RSD, %).

| weeks | P1 | P3 | P4 | P5 | P6 | P7 |
|---|---|---|---|---|---|---|
| before | 241 (76%)* | 463 (53%)* | 300 (0%)*/** | 159 (57%)** | —** | 18 (46%) |
| 8 | n.d. | | | 124 (50%) | — | 99 (125%) |
| 20 | 132 (104%) | | 80 (90%) | 141 (75%) | — | 41 (191%) |
| 30 | n.d. | | 71 (123%) | | | 87 (76%) |
| 40 | n.d. | | 58 (45%) | | | |
| 50 | 38 (68%) | | 55 (97%) | | | |
| 60 | n.d. | | 70 (97%) | | | |
| 70 | n.d. | 30 (37%) | | | | |
| 80 | 12 (108%) | 28 (26%) | | | | |
| 90 | 12 (96%) | 23 (29%) | | | | |
| 100 | 26 (80%) | 40 (50%) | | | | |
| 110 | 26 (74%) | 46 (25%) | | | | |
| 120 | 26 (73%) | 60 (30%) | | | | |
| 130 | 31 (48%) | | | | | |
| 140 | 33 (24%) | | | | | |
| 150 | 20 (54%) | | | | | |

| weeks | P8 | P9 | P11 | P12 | Mean | |
|---|---|---|---|---|---|---|
| before | 29 (95%) | 51 (61%) | 67 (66%)** | 46 (67%) | 152 | (108%) |
| 8 | 8 (72%) | 136 (63%) | 143 (54%) | 90 (63%) | 95 | (57%) |
| 20 | 11 (36%) | | 152 (57%) | 111 (62%) | 87 | (62%) |
| 30 | 7 (90%) | | | | 55 | (77%) |
| 40 | | | | | 58 | (—) |
| 50 | | | | | 47 | (26%) |
| 60 | | | | | 50 | (—) |
| 70 | | | | | 30 | (—) |
| 80 | | | | | 20 | (57%) |
| 90 | | | | | 18 | (44%) |
| 100 | | | | | 33 | (29%) |
| 110 | | | | | 36 | (39%) |
| 120 | | | | | 43 | (56%) |
| 130 | | | | | 31 | (—) |
| 140 | | | | | 33 | (—) |
| 150 | | | | | 20 | (—) |

*estimated from dairies or medical journals;
**with sleep medicine. For P6 no data was possible to obtain because of low communication ability.

Four persons (P7, P9, P11 and P12) experienced worsening in SOL during first 8-30 weeks (Tab. 8). One of them used sleep medication before.

Number of Awakenings and Minutes Awake During Night Sleep

The sleep patterns of many ME patients are dramatically disturbed. The main features during the night are increased pain intensity and duration (symptom 4 on the List of Canadian Criteria); palpitations (tachycardia, 6Ab); difficulty breathing (6Ad); frequent nocturnal urination (6Ae) and night sweats/freezing (6Ba). Those can explain the increase in next parameter—number of awakening (NWAK) and period without sleep (WASO). One of the most extreme examples was documented for P1 before and during first 20 weeks of treatment.

Visualization of improvement demonstrates the decrease in all measured parameters, such as number of awakenings (NWAK), wake after sleep onset (WASO), reduction in numbers of urinations and pain severity. Number of awakenings before treatment was 4.6±8.1 times per night for 9 of 10 persons, with maximum 22 (P3) and minimum 0.1 (P9, P11) awakenings. Two of 10 persons had profound disturbance of NWAK, which was back to normal during first 8-20 weeks of treatment. Number of minutes wake after sleep onset (WASO) was in the range of 512 to 3 minutes. Improvement of this parameter was documented during first 8-20 weeks of treatment to range of 50-0 minutes for all group.

The decrease in Mean number of awakenings per night during self-treatment course is shown in FIG. 13.

Sleep Efficiency (%)

Percentage of time spent asleep from the amount of time spent in bed (TST/TIB*100) is the parameter called sleep efficiency. Improvement of the parameters NWAK, WASO, SOL together with symptoms (6Ab,d,e and 6Ba) at the same time as TiB was not significantly changed, altogether resulted in dramatic improvement of the night sleep as shown in Tab. 10. Due to large interindividual differences in condition severity, also individually improvements are shown in FIG. 14.

TABLE 10
Sleep Efficiency (%), mean of 14 consecutive nights (±S.D.).

| weeks | P1 | | P3 | P4 | P5 | P6 | P7 | P8 | P9 | P11 | P12 |
|---|---|---|---|---|---|---|---|---|---|---|---|
| before | 35 | (16)* | 9 (13)* | 29 (—)*/** | 52 (20)** | —** | 89 (13) | 77 (17) | 38 (46) | 85 (10)** | 82 (17) |
| 8 | 60 | (30) | | | 62 (8) | — | 66 (39) | 92 (3) | 23 (33) | 67 (15) | 79 (13) |
| 20 | 83 | (15) | | 77 (17) | 41 (13) | — | 88 (15) | 88 (5) | | 69 (19) | 70 (21) |
| 30 | 92 | (10) | | 80 (18) | | | 77 (15) | 94 (3) | | | |
| 40 | n.d. | | | 80 (9) | | | | | | | |
| 50 | 90 | (4) | | 82 (17) | | | | | | | |
| 60 | n.d. | | | 79 (15) | | | | | | | |
| 70 | n.d. | | 75 (20) | | | | | | | | |
| 80 | 98 | (3) | 86 (10) | | | | | | | | |
| 90 | 98 | (2) | 82 (11) | | | | | | | | |
| 100 | 96 | (5) | 81 (15) | | | | | | | | |
| 110 | 93 | (2) | 79 (9) | | | | | | | | |
| 120 | 90 | (11) | 76 (11) | | | | | | | | |
| 130 | 84 | (8) | | | | | | | | | |
| 140 | 91 | (5) | | | | | | | | | |
| 150 | 95 | (3) | | | | | | | | | |

*estimated from dairies or medical journals;
**with sleep medicine. For P6 no data was possible to obtain because of low communication ability.

Sleep efficiency varied between persons at the beginning: one person was not monitored for sleep parameters (P6). Five of 10 persons had severe sleep disturbance with sleep efficiency in the range of 9 to 52%: P1, 3, 4, 5, and 9. Four of 10 persons had acceptable level of sleep efficiency in the range of 77 to 89%: P7,8,11 and 12. Improvement of the condition at least by 47% was achieved in P1, P3 (personal communication), P4 during first 20 weeks and by 17% in P8 (Tab.9). Altogether, in four of 10 persons. Sleep efficiency in persons who used sleep medicine (P5 and P11) was still in adjustment and it is necessarily to emphasize that full dose of the treatment was just introduced and time was needed to achieve the desired effect. Five of 10 persons did not experienced improvement during first 8-20 weeks.

However, they sleep disturbances were not severe, except for P9 who did not received full dose at 8 weeks. Effect documented for persons with severe disturbances (P1, P3, P4) and P8 lasted for more than 1 year.

### EXAMPLE 3—DIAGNOSIS OF CFS/ME/SEID

We have shown that there is a correlation between the increased lactate level in blood and diagnosis of CFS/ME/SEID. Total lactate Load during 155 min was 44% higher in persons diagnosed with CFS/ME/SEID compared to persons not suspected for disease. Thus, diagnosis given only when Canadian Criteria (2003) are fulfilled and Canadian Criteria are the list of the symptoms, including fatigue presence and severity and cognitive functioning, we may deduce that increase lactate level in blood associated with symptom burden, fatigue presence and cognitive functioning, and this can be used to diagnose and monitor the development of diagnosis CFS/ME/SEID. We also had used this test to distinguish between the real and placebo effect (Example 5—Placebo-test). The example of such monitoring and association between lactate levels and symptoms shown in another example.

Normal values of lactate in human blood is within the range of 0.5 to 2.0 mmol/L. Lactate clearance in healthy persons is organised via Gori cycle in liver (60% of clearance), kidneys (30%) and other organs and tissues, such as heart, muscles, intestine. During Gori cycle, lactate is transported from working organs and muscles into liver cells, where it transforms to pyruvate, which then stored in a form of glucose. Glucose, if it is needs for energy, is transported back to muscles or other activated tissues, when it undergoes chemical transformations to give rise to the key compounds of energy producing TCA cycle. The energy stored in form of phosphate bonds of ATP molecules. 38 (36) molecules of ATP are produced from two glucose molecules during TCA cycle. Released from phosphate bonds energy, used for cognitive and physical performances (job) of the body.

Monitoring of lactate levels in a routine clinical practice is not widely spread, probably because: (1) it is presents in all cells, except for the mature erythrocytes, which do not have mitochondria; (2) levels are increased under physical exercises; (3) levels are normally very strictly regulated, and abnormal levels associated with life threatening conditions are only slightly above the normal range. However, some health conditions, where monitoring of lactate levels are useful, could be listed. They are all associated with hyperlactatemia. Hyperlactatemia is life treating condition, when levels of lactate in a blood >2.0 mmol/L with or without the presence of lactic acidosis or with or without the disturbed in lactate-to-pyruvate ratio.

In several clinical conditions, admission hyperlactataemia has been demonstrated to be a predictor of death or outcome in: children with sepsis (Duke et al., 1997); cardiac surgical patients (Khosravani et al., 2009); trauma/neuro patients (Khosravani et al., 2009); shock or respiratory and renal failure (Juneja et al., 2005); rupted abdominal aortic aneurysm repaired (Singhal et al., 2005), to mention some. Another group of conditions where hyperlactatemia is direct course of the death is innate hereditary metabolic disorders. Some examples are: mitochondrial encephalomyopathy, pyruvate carboxylase deficiency; succinyl-CoA ligase deficiency; mitochondrial hepatoencephalomyopathy. Course of the death at such conditions is decreased clearance or increased production of the lactate as a result of biochemical disturbances in pathways or as result of misbalance caused by treatment.

To our knowledge, the medicinal pharmacological treatment against hyperlactatemia (with or without lactic acidosis and disturbed lactate-to-pyruvate ratio) does not exist. The widely spread clinical practice described in literature is called “Lactate guided therapy”. Lactate guided therapy has following therapeutic targets: optimizing mean arterial pressure by fluid isotope administration; multi-organ protection by early enteral nutrition; optimizing metabolic control by means of insulin infusion therapy; prevention/treatment of infections.

Individuals affected with CFS/SEID/ME experience the extremely and instantaneously unpleasant features in response to daily life activities, such as “unwell after eating”, “inner shackles”, “trembling”, “extremely dizziness during conversation”, “nausea”, “sweating”, “increase heart rate”, “palpitations”, “bellyache”, “extremely fatigue” and “instantaneous demand of rest or sleep” to mention some. All of them may occur simultaneously and after normal physical movements and functions (such as eating, listening, etc.) and/or daily life cognitive tasks (such as writing a short notice, read text or coking recipe).

We have shown that those unpleasant features are associated with fluctuations in lactate levels. The parameters associated with the lactate level can be used for functional diagnostics of CFS/ME/SEIS.

We show that lactate can be a biomarker for the ME/CFS/SEID. It is important how to measure it and what to calculate. Through this method (1) abnormal Basal Housekeeping Lactate Levels (BHLL) shown in persons, who is not moving and thus without any contribution from the muscle job or anaerobe threshold; (2) typically Instantaneous Lactate Fluctuations (ILF) during given time visualised by lactatogram; (3) and Total Lactate Load (TLL) of the capillary blood can be calculated as Area Under the Curve (AUC) for measurements during 155 minutes; (4) additionally measurements in extremities visualise the physiological defence mechanism—to remove the damaging metabolites away from brain, heart and lungs. Standard deviations of all mentioned values are also important diagnostic criteria, which mirrors the fluctuations of the levels within a given data set and thus reflecting how unpleasant the patient have it.

Description of the Lactate Measurements (Lactogram)

The test person with suspicion for ME/CFS/SEID should avoid hard physical or cognitive activity (for example, as preparation to exam or examination, holding presentations and such) at least for 24 hours prior to the test. Light meal could be eaten minimum 60 minutes before the test. Test persons for reference group (not suspicion for ME/CFS/SEID) should have a normal working and activity day and drive a can car to the test, if desired. Test person lies in the bed or sofa under the entire test, in a quiet, good ventilated room at ambient room temperature. Blanket or hot water bottle could be used on feet, if desired. In this test, healthy persons were also allowed to talk under the test, to introduce as much stimuli as possible, however, ME test persons were all severely affected and were not able to talk.

First 60 minutes of the measurement gives presentation of the basal lactate levels, following by the cognitive provocation by reading of a demanding text in two steps—10 min and 5 min, with 75 minutes of relaxation afterwards. To give a physiological stimuli (eating and digestion), the light meal of banana, toast or glass a water had been given to test person between 10-20 minutes post reading. The produced graphic presentation of the lactate levels we called lactatogram.

Lactate levels are measured by mobile tester Lactate Scout+ from EKF Diagnostics (Germany) for self-monitoring. Fingertip was cleaned, dried and punctured by Microlet lancett fra Bayer (Germany) and 0.2 whole capillary blood was absorbed into Lactate Scout Sensors from SensLab GmbH (Germany).

Results were manually registered in electronic format, with additional confirmation by screenshots of the monitor by iPhone-6S.

The monitoring was undertaken each 5^{th} minute, which some 10 minutes intervals, to avoid unknown periodic cycles in the lactate production. Example of a typical lactatogram in a healthy person is shown on FIG. 15.

Participants

Totally thirty randomly chosen persons voluntarily participated in the test, where 10 were affected by ME/CFS/SEID and had authorised diagnosis based on the Canadian Criteria (2003) and 20 persons recognised them self as healthy.

Basic statistics was performed in Excel from Office 365, including T-test. AUC was calculated according to the trapezoid method:

$${\int }_{s}^{b} f ( t ) dt = {\sum }_{k = 1}^{N} \frac{f ( {t}_{k - 1} ) + f ( {t}_{k} )}{2} {Δt}_{k}$$

were tₖ is the time when the k-th measure was performed and f(tₖ) is the corresponding concentration value, i.e. the first measure of the concentration value f(t₀) is taken at the time t₀, the next f(t₁) is taken at the time t₁, incremented until the last measure f(t_{N}) is taken at the time t_{N}. Δtₖ=tₖ−tₖ₋₁ is the length of the k-th subinterval, i.e. time difference between two measures k and k−1.

Healthy Individuals

Typical Lactatogram in healthy person is presented on FIG. 15 and all produced in this test lactatograms are shown on FIG. 16. Under the measurements in healthy group we wanted to introduce as much stimuli as possible, that why all participants get light meal between 10-20 minutes post reading. None special consideration was given to any diagnosis, which is not ME/CFS/SEID and all participants were randomly chosen.

TABLE 11
Parameters of lactatogram in healthy individuals.

| | Lactatograms of Healthy persons | | | | | |
|---|---|---|---|---|---|---|
| | Min | Max | Mean (range) | Mean of all | S.D. | R.S.D. (%) |
| High (absolute value), | 0.5 | 6.1 | 0.7-2.0 | 1.4 | 0.3 | 24 |
| mmol/L | | | | | | |
| AUC₁₅₅, | 105.3 | 317.3 | 105.3-317.3 | 212.3 | 45 | 21 |
| mmol/L*min | | | | | | |

Expected calculated AUC₁₅₅ if all measurements were as bottom normal boarder line of 0.5 mmol/L was 77.5 and 310 mmol/L*min, if all measurements were as upper normal boarder line of 2.0 mmol/L lactate. AUC₁₅₅ of lactate levels in randomly chosen healthy population (N=20) was 212.3±45 mmol/L*min. Single high absolute values did not influence the AUC, as long they are occurring once or twice during 155 minutes of the test.

Basal Housekeeping Lactate Levels (BHLL), as appeared on lactatograms, fluctuated within the normal range of 0.5 to 2.0 mmol/L. However, some increased single absolute values were observed after provocation by reading and eating, also in healthy persons. The Instantaneous Lactate Fluctuations (ILF) have been registered in 7 of 20 persons, with the largest rise between following measurements of 2.6 mmol/L in none-smokers and 3.7 mmol/L in heavily smoking person (I.D.29). Total Lactate Load (TLL) of the capillary blood was calculated as AUC₁₅₅, Table 12.

Additionally, Lactate in Extremities (LE) measured at the end of the test did not unveiled any extremely single values (FIG. 20). All persons performed the test after their routine working day, including driving the car, cognitive or physical work, communication and responsibilities toward the family and children. Despite of it, all Basal Housekeeping Lactate Levels were low, with moderate increase in feet (Table 12). This mean that: (1) in people with normal lactate metabolism no unexplained lactate abundance was detected in extremities; (2) people who performed body job or cognitive effort day before the test had higher lactate levels in feet but not in the capillary blood of fingers; (3) lactate levels were equally distributed between left and right side (FIG. 20).

TABLE 12
Lactate measurements.

| | Basal | | | | | Mean | | |
|---|---|---|---|---|---|---|---|---|
| | Housekeeping | | | | | Lactate in | Total | |
| | Mean Lactate | | | | | Extremities | Lactate | |
| | Levels, | | | | | (LE), | Load₁₅₅ | |
| | mmol/L, | Hallux | | Fingertip | | mmol/L | (AUC), | Gender, |
| I.D. | (S.D.) | right | left | right | left | (S.D.) | mmol/L*min | age |
| 12 | 1.3 (0.4) | 1.2 | 1.7 | 1.4 | 1.2 | 1.4 (0.2) | 200.0 | M, 34 |
| 13 | 0.9 (0.3) | 3.0* | 3.3 | 1.5 | 1.5 | 2.3 (1.0) | 145.5 | M, 53 |
| 14 | 1.6 (0.4) | 1.3 | 1.8 | 1.6 | 1.1 | 1.5 (0.3) | 243.0 | F, 52 |
| 15 | 1.2 (0.3) | 1.8 | 1.5 | 1.2 | 0.5 | 1.3 (0.5) | 195.8 | M, 48 |
| 16 | 1.1 (0.4) | 3.4* | 2.7 | 0.7 | 1.5 | 2.1 (1.2) | 171.3 | M, 49 |
| 17 | 0.7 (0.2) | 1.3 | 0.8 | 0.7 | 0.7 | 0.9 (0.3) | 85.8 | F, 56 |
| 18 | 2.0 (0.8) | 3.3 | 2.9 | 1.0 | 0.8 | 2.0 (1.3) | 251.3 | F, 43 |
| 19 | 1.6 (0.9) | 1.6 | 4.5*** | 0.7 | 0.5 | 1.8 (1.8) | 198.5 | F, 39 |
| 20 | 1.5 (0.5) | 1.6 | 1.6 | 1.2 | 1.3 | 1.4 (0.2) | 181.0 | M, 35 |
| 21 | 1.3 (0.4) | 1.4 | 1.1 | 0.7 | 0.7 | 1.0 (0.3) | 161.8 | F, 38 |
| 22 | 1.4 (0.7) | 5.0 | 4.1 | 1.1 | 1.7 | 3.0 (1.9)*** | 169.5 | F, 26 |
| 23 | 1.7 (0.7) | 1.6 | 1.2 | 1.5 | 2.7 | 1.8 (0.7) | 273.5 | M, 21, AN |
| 25 | 0.9 (0.4) | 3.3** | 2.6 | 1.6 | 0.8 | 2.1 (1.1) | 171.8 | F, 27 |
| 26 | 1.3 (0.4) | 1.7 | 1.6 | 2.1 | 1.0 | 1.6 (0.5) | 198.8 | F, 42, HYPO |
| 27 | 1.5 (0.4) | 2.5 | 2.2 | 1.0 | 2.0 | 1.9 (0.7) | 232.3 | M, 46, SM |
| 28 | 1.6 (0.4) | 1.6 | 2.2 | 1.1 | 1.3 | 1.6 (0.5) | 247.3 | F, 42 |
| 29 | 1.8 (1.2) | 1.3 | 1.6 | 1.7 | 1.1 | 1.4 (0.3) | 268.8 | M, 67, SM |
| 31 | 1.5 (0.7) | 1.7 | 1.4 | 2.3 | 1.5 | 1.7 (0.4) | 186.5 | F, 22 |
| 32 | 1.5 (0.5) | 2.4 | 2.4 | 1.7 | 1.0 | 1.9 (0.7) | 192.3 | F, 24 |
| 33 | 1.7 (0.3) | 2.3 | 2.0 | 1.7 | 1.8 | 2.0 (0.3) | 262.8 | F, 62, HYPO |

*person who despite the instructions run less than 12 hours before the test with speed 12 km/h for 60 minutes;
**person who despite of the instructions read to exam for 12 hours during day before the test.
***this person experiencing scatting and burning pain in her right foot and leg, with profound pain in foot sole on the daily basis for last 50 weeks, no cause is found.
****this person had a trauma in ankle of the right foot and it looks like lactate was led away from the traumatised foot. AN = antibiotics, HYPO = hypotheriose, SM = smoker or sniffer of tobacco.

Patients with CFS/ME/SEID

FIG. 17 demonstrates comparative lactatograms of healthy person and two persons affected by ME. Basal Housekeeping Lactate Levels (BHLL) of persons with ME were slightly (in grey) or significantly (in green) higher than in healthy person (in orange). Instantaneous Lactate Fluctuations (ILF) during given time were more profound in persons with ME, and single arise/decrease within 5 minutes intervals were in the range of 3.2-20.2 mmol/L (from 2.5 to 5.7 and back to 3.3 mmol/L or from 4.5 to 24.7 and back to 1.9 mmol/L). In comparison, corresponding values in healthy population were 2.6 mmol/L and 3.7 mmol/L for smoker. This is much more steep and unpleasant in persons with ME/CFS/SEID. ILFs were provoked by reading and eating (grey line) as well as were observed before provocation (in green). Those measurements were in good agreement with persons individual perception of own condition: sweating, increased heart rate, breathing problems, pains and other symptoms from the list of Canadian Criteria.

The Total Lactate Load (TLL) of the capillary blood in all persons affect by ME/SEID/CFS are shown in FIG. 18. All lactatograms have same features as described above. The AUC (Table 13) were in the range 259.5 to 569.5 mmol/L*min. Highest absolute value measured was 24.7 mmol/L.

TABLE 13
Parameters of lactatogram in individuals with authorized ME/CFS/SEID
diagnosis according to Canadian Criteria (2003).

| | Lactatograms of persons affected by CFS/SEID/ME | | | | | |
|---|---|---|---|---|---|---|
| | Min | Max | Mean (range) | Mean of all | S.D. | R.S.D. (%) |
| High (absolute value), mmol/L | 0.5 | 24.7 | 1.7-3.5 | 2.3 | 1.1 | 46 |
| AUC₁₅₅, mmol/L*min | 259.3 | 569.5 | 259.5-569.5 | 380.0 | 112.2 | 32 |

Measurements of the Lactate in Extremities (Table 14 and FIG. 20) had shown characteristics pattern: highest values in left foot (hallux) and right hand (fingertip). The lowest and highest absolute values were 1.6 and 12.1 mmol/L in right foot and 2.5 and 18.7 mmol/L in left foot, while corresponding values in right and left hand were 0.9 and 12.9 mmol/L against 1.4 and 10.0 mmol/L. Standard deviations of Basal Housekeeping Mean Lactate level were in the range of 0.8 to 4.7 and for mean Lactate in Extremities—0.6 to 7.6. Thus, standard deviation corresponds for the variation within a data set, in this context it has meaning of how instantaneously levels of lactate are changing within a given period of time, what in next turn associates with unpleasant experiences and symptom appearance.

TABLE 14
Lactate measurements in ME/CFS/SEID patients. None of the persons
were allowed or could not speak under the test because of severe
condition. This means that external stimuli were naturally minimized
in comparison to the group of healthy volunteers.

| | Basal | | | | | Mean | | |
|---|---|---|---|---|---|---|---|---|
| | Housekeeping | | | | | Lactate in | Total | |
| | Mean Lactate | | | | | Extremities | Lactate | |
| | Levels, | | | | | (LE), | Load₁₅₅ | |
| | mmol/L, | Hallux | | Fingertip | | mmol/L | (AUC), | |
| I.D. | (S.D.) | right | left | right | left | (S.D.) | mmol/L*min | Gender |
| 1 | 3.0 (1.0) | 9.7 | 18.7 | 2.7 | 2.5 | 8.4 (7.6) | 464.8 | F, 48 |
| 2 | 2.7 (1.1) | 2.7 | 2.9 | 3.9 | 2.6 | 3.0 (0.6) | 405.8 | M, 18 |
| 3 | 1.8 (0.8) | 7.7 | 4.9 | 1.7 | 2.5 | 3.9 (3.1) | 268.0 | M, 30 |
| 5 | n.d. | 5.7 | 5.0 | 3.0 | 1.4 | 3.8 (2.0) | n.d. | F, 60 |
| 6 | 1.7 (2.3) | 12.1 | 13.4 | 12.9 | 10.0 | 12.1 (1.5) | 259.3 | M, 40 |
| 7 | 3.5 (1.8) | 3.1 | 11.3 | 4.5 | 2.2 | 5.3 (4.1) | 534.0 | F, 42 |
| 8 | 3.5 (3.2) | 1.6 | 2.5 | 1.2 | 1.4 | 1.7 (0.6) | 569.5 | F, 40 |
| 9 | 1.8 (0.9) | n.d. | n.d. | n.d. | n.d. | n.d. | 267.8 | F, 40 |
| 11 | 1.8 (0.8) | 2.4 | 2.8 | 2.1 | 1.9 | 2.3 (0.4) | 266.8 | F, 32 |
| 12 | 1.8 (1.1) | 1.7 | 5.2 | 7.1 | 1.5 | 3.9 (2.7) | 298.3 | F, 46 |
| 13 | 3.1 (4.7) | 7.0 | 7.2 | 0.9 | 2.2 | 4.3 (3.2) | 466.0 | M, 21 |

T-Test of Total Lactate Load (AUC₁₅₅) in ME(CFS/SEID Patients Vs Normal Population

Measurements of capillary lactate was performed in double as many healthy persons (N=20) as those with ME (N=10). Because, the occurrence of ME/CFS/SEID is approximately, 1-2% in population, the chance to found patients without diagnosis, but affected by ME/CFS/SEID was considered to be minimal. T-test of ME group Total Lactate Load (AUC) against respectively, Healthy group, Healthy women and Healthy men showed statistically significant difference at P<0.05 (Table 15). There were no significant differences in Total Lactate Load (AUC) between genders in either ME or Healthy group. Generally, in clinical practice, the pathological values considered to be are those increased for >20% of value in normal population (reference value). Reported here, Total Lactate Load in patients with diagnosis ME/CFS/SEID was increased by 44% of respective value measured in healthy people (FIG. 19 and Table 15). Analytical uncertainty of the test was determined to be: 2.9% for 10 consecutive measurements during same day and 3.8% for 7 consecutive days.

TABLE 15
Comparison of mean Total Lactate Load₁₅₅
(AUC) in ME group and normal population.

| | | | | ME all, | ME women, | ME men, |
|---|---|---|---|---|---|---|
| | mean | | | T-test | T-test | T-test |
| | AUC₁₅₅ | S.D. | RSD | P-value | P-value | P-value |
| ME all, N = 10 | 380 | 122 | 32 | | | |
| Healthy, N = 19 | 213 | 46 | 22 | 0.00088 | | |
| ME women, N = 6 | 400 | 139 | 35 | | | |
| Healthy women, N = 11 | 212 | 49 | 23 | 0.00085 | 0.00995 | |
| ME men, N = 4 | 350 | 103 | 29 | | | |
| Healthy man, N = 8 | 214 | 45 | 21 | 0.00093 | | 0.03580 |
| T. Normal min | 78 | — | — | | | |
| T. Normal max | 310 | — | — | | | |

To perform the test and build up the lactatogram 26 measurements were undertaken during 155 min of the test. Standard deviation for each of 20 set (N=20) consisting of 26 measurements each was 0.2 to 1.2 mmol/L (Table 12) in healthy population and 0.8 to 4.7 mmol/L (Table 14) in group of ME/SEID/CFS affected. This is a one more parameter, which should be considered when tests results are reviewed for diagnosis.

FIG. 20 demonstrate the comparison of the Lactate in Extremities (LE) in both groups. In general, the differences between groups were associated with two main features: (1) absolute values and (2) bilateral differences or asymmetry in lactate distribution. There was no difference in absolute values of hallux (foot) or fingertips (hand) on both sides of the body, however the absolute mean value in fingertips were a half of those in hallux in healthy group.

In ME/CFS/SEID affected persons the absolute mean values were up to 4 times higher than in healthy persons. Profound difference in right and left body sides was unveiled as an unique pattern: highest in left hallux and right fingertip. Therefore, all performed tests were designed according to this pattern: left hallux (foot) and fingertips on right hand (hand).

Conclusion: We are the first to demonstrate the fluctuations of lactate levels (Basal Housekeeping Lactate Levels (BHLL)) in all living persons under light cognitive (reading) and physiological activity (eating). In healthy persons, the fluctuations are in general within the normal range of 0.5 to 2.0 mmol/L. Standard deviations of 26 measurements during 155 min are higher in persons affected by ME/CFS/SEID and might be associated and explain the instantaneously need for rest and sleep and other unpleasant experiences or symptoms.

Following parameters are recommended to be considered for diagnostics:
Range of absolute values;
AUC₁₅₅
Standard deviation for 26 consecutive measurements during 155 min of test;
Sloop (steepness) for instantaneously Lactate Fluctuations;
Characteristic pattern of lactate distribution in extremities.

### EXAMPLE 4—TEMPORARY LOSS OF SIGHT IN RESPONSE TO LOWERING OF CARBOHYDRATES IN DRINKABLE

Some ME/SEID/CFS patients are also suffering of cancer. Cancer diet often characterized by reduction of carbohydrates (sugars), which faster increase glucose level in the blood. However, it is right or wrong, some patients keep this diet and argue for that the large amounts of available sugars are stimulate the cancer growth. Because of it, the objective of this test was to examine if reduction of carbohydrates (sugars) will affect the therapeutic effect of the drinkable.

Drinkable was made to meet a demand of 153 mg soluble oxalates and the same nutritional value as shown in Tab.1 in example 1, with one minor deviation—the total amount of sugars should be reduced by 25%, what corresponds to 6.5 g. The rest of the sugar mixture volume was exchanged with distilled, deionised water. This drinkable with reduced amount of sugars will be called “reduced” and had the following contains of the carbohydrates (g in 250 ml portion): sucrose—0.4; lactose—<0.7; maltose —<0.7; glucose—6.4; fructose—12.1 and sum of all—19.2 g, as calculated from Tab. 1. Blood glucose was monitored during whole accident, from fingertips, by self-monitoring apparatus from Bayer (Germany) by a second person. Measurements were started 10 minutes after intake and performed as following: for first 5 minutes—each minute, then each 5^{th} and at the end each 10th minutes.

The 250 ml of reduced drinkable was consumed, when within 3-5 minutes later P1 could not see another person in a doorway, as visualised on the FIG. 21. Sight came and went back and forth. Within seconds additional symptoms came, such as powerful explosions of orange colour and similar to the fireworks. Rest of the field of view had disappeared behind a blanket: first, sides fields on both eyes, afterwards large areas in the middle of the field of view. It resulted that only some parts of another person could be seen.

The volume of sugar, equal to that reduced % was given immediately to P1, only 12 minutes after sight problems started. Sight continued to disappear as described below, in addition to loss of ½ of a field of view as shown on FIG. 21. The dense grey blanket was not possible to remove either with eye or head movements.

Described worsening continued for total 25 min, when the symptoms finally get into revers and started to disappear in an opposite order: first disappeared grey blanket, then picture in the middle of the field of view came back, afterwards came sides of field of view. Some bodily reactions continued for 4 hours. After that P1 was hospitalised, but none pathologic findings or neurological disorders were unveiled on the CT of the head, eye and nerves examination.

There was not found any extreme glucose levels in capillary blood (FIG. 21). P1 had eaten lunch 45 minutes before intake of the reduced drinkable. The sight was restored 25 minutes post intake of drinkable. At this period of 25 min glucose levels were within the range of 5.6 to 6.7 mmol/L, which is normal level. In the period when glucose levels increased within the range of 6.4 to 8.3 mmol/L, also sight symptoms had disappeared. It is necessarily to remain that 6.5 g carbohydrate mixture was given at 12 minutes post intake of the drinkable and thus, might be responsible for increase of the blood glucose.

It can be concluded from this example that sufficient amount of carbohydrates (sugars) should be used in combination with soluble oxalates in drinkable to avoid extreme symptoms worsening.

The drinkable solutions must thus either be mixed of ingredients containing the sufficient amount of sugar, or a mix of carbohydrates (sugars) can be added to the drinkable in a same composition (ratio) and dose as in Tab. 1.

### EXAMPLE 5—MONITORING OF HEALTH IMPROVEMENT DURING THE COURSE OF SELF-TREATMENT OF CFS/ME/SEID WITH THE HELP OF LACTATE MEASUREMENTS

Effect of Bolus—Case A

Reading test (Lactatogram for 155 min) was taken as previously described: before treatment (FIG. 22). Person (P8) arrived the test in a car as driver both times, what was against the instructions. Whatever it is the stimuli of driving or severe ME condition had caused extremely high basal levels of lactate will remain unveiled. In comparison, no such increase was detected in any of 20 healthy persons attended the test by own car (described elsewhere). This means that healthy persons regulate lactate levels within the normal range: either cognitive effort (driving, reading) or eating do not lead to increase.

Extremely high absolute values before and under the reading test appeared on Lactatogram of P8 (FIG. 22). Pain in the head, foggy brain and pressure into the head, was the most profound symptoms at that point of time. To unveil the immediate effect of drinkable oxalates on reducing lactate levels, P8 was offered the consumption of 250 ml active drinkable on 10^{th} minute post fulfilled reading. P8 reported improvement in condition and vanishing of all named symptoms within 10 minutes post intake, simultaneously as basal lactate levels dropped from 6.2 to 0.5 mmol/L. The low lactate levels remained within normal range during following 70 minutes of the test (except for one measurement at 65 min post reading).

Increased Levels of Lactate as Result of Stopped Treatment at Week 120—Case B

Basal levels of lactate were measured during three days before treatment was totally stopped, and measurements performed on day 1, 6 and 7 without the treatment, and on day 1 and 2 after restoration of a treatment. All measurements were performed at the same time-points ±5 min, randomly chosen first day of experiment. Daily activity was kept identical within all four initial days and can be associated with activity at week 120.

Absolute lactate levels in capillary blood were within the normal range of 0.5 to 2.0 mmol/L, for 43 consecutive measurements during 72 hours prior to treatment stop, except for 1 value above—2.3 mmol/L at 2377 min (FIG. 23). The range for the measurements on treatment was 0.8-2.3 mmol/L, and raised to 1.0-3.0 mmol/L during first day without a treatment, and to 1.2-7.1 mmol/L during totally 7 days without treatment. Those values are fare higher than normal range (described elsewhere). Absence of a treatment caused severe worsening of the health condition: during 7 days P1 became bed-bound with dramatically extended scope and severity of symptoms (§ 2) occurred simultaneously. P1 was not able to leave the bed for urination, had to be feed. Lactate levels get lower almost immediately after the treatment with full dose was re-established (FIG. 23), with the range of 1.2-3.5 mmol/L. Improvements, such as, decrease in symptoms burden, pain relief and decreased fatigue came along with the depicted lowering of the lactate levels.

Conclusion: Based on the observations in Case A and B, the association between clinical picture, symptoms scope and severity and measured levels of capillary lactate had been established. The Lactatograms and point measurements in extremities will be further used as one of the parameters in monitoring of health improvement during self-treatment course.

Decrease in Total Lactate Load, Absolute Lactate Values, Lactate in Extremities and Standard Deviations as a Result of Self-Treatment (N=6)

Lactatograms were taken as described elsewhere: before start and at 20 weeks of treatment. Point measurements in extremities had been performed at the same time as Lactatograms. Validation of the Lactatograms was performed on three parameters: (1) Absolute values of Lactate in capillary blood; (2) Total Lactate Load measured as AUC₁₅₅; (3) Standard deviation in full set of 26 measurements. Measurements in extremities were validated on three parameters: (1) Absolute values in each extremity; (2) Mean of all 4 measurements (right foot, left foot, right hand and left hand); (3) Standard deviation for 4 measurements.

Absolute values of lactate correspond to the real-time fluctuations of lactate and should be compared with normal range, which is 0.5-2.0 mmol/L. Total Lactate Load gives a picture of lactate fluctuations and levels during consecutive 155 min and reflect the symptom related experience and perception of person's own health form under the test. Since standard deviation describes variety within a data set, the size of it gives association of how unpleasant person felt during 155 min of monitoring. All mentioned above parameters thought to correspond to the fast-circulating and/or newly produced lactate, in same manner as glucose measurements used in diabetics. In contrary, overload of lactate was found in lower extremities as described elsewhere, away from important organs such as heart and brain. Therefore, measurements in extremities give a picture of lasting lactate or lactate load over the time, in the same manner as HbA₁C used in monitoring of the diabetics.

Six of eleven persons were tested: P1, P3, P7, P8, P11 and P12. Two of eleven had terminated treatment or did not want to be measured (P5, P6). Three of eleven started treatment long before test was developed and therefore were not measured at all (P2) or some parameters are missing (P1, P3). One of eleven persons had severe condition and could not be tested yet (P4). One of eleven persons dis not came to 20 weeks yet (P9).

Four parameters were calculated from 26 points on Lactatogram and 4 measurements in extremities. Improvement of the 4 of 4 measured parameters in P8 shown on FIG. 22 and where in compliance with each other—Absolute Values and Total Lactate Load (AUC₁₅₅), both decreased by 64-67%. Decreased Standard deviations for both mentioned parameters were in compliance with reported health improvement during 20 weeks (Tab. 21), as decrease in symptom number from 21 to 11 and increase of CEDL from 10.6 to 30.6 (Tab. 22). Lowered mean value of lactate in extremities (lasting lactate in the body) was also reduced in the same magnitude—by 64%, reflecting the persistent effect of the treatment on lowering of lactate load over the time.

Decrease by same magnitude of 35% was documented for all parameters in P7—Absolute Value of capillary lactate, Total Lactate Load, Lactate in extremities, along with decreased values of standard deviations for all parameters (Table 16), simultaneously as number of symptoms decreased from 21 to 15 (Tab.21) during 20 weeks of treatment. CEDL validation was compromised by change to walk by own force instead of using walking chair (Tab.22).

Decrease by 15% was detected in P11 in Lactate in Extremities, corresponding to reduction of lactate load over the time. Lactate values were in good compliance with reported lowering of symptom number from 22 to 16 (Table 21) and increase of CEDL from 0.80 to 1.03 (Table 22) during 20 weeks of treatment. Unexpected increases in Total Lactate Load and mean of Absolute capillary values were documented in P11, as only one of six measured persons, and was 16%. For instance, analytical day-to-day variation of the test apparatus was calculated to be 3.8% for 7 consecutive days and thus, cannot explain this increase alone. However, the person itself explain the difference by unusually god form on a test day before the treatment.

Decrease in P3 by 45% was documented for Lactate in Extremities.

The reduction of 47% for Lactate in Extremities for P12 coincidence with increased of the CEDL from 3.99 to 7.26 during 20 weeks of treatment (Table 22). It might have indicated that values of lasting lactate have some association to fatigue and stamina perception and experience. The modest reduction of 8% in Total Lactate Load coincidence to absence of reduction in symptom number during those 20 weeks (Table 21): 10 and 11 reported symptoms left at both time points. On another hand, the decrease in mean of Absolute capillary values and Standard deviations of all parameters, might evidence for less variation in measured values and more pleasant daily experience.

The special attention should be draw to parameter such as Lactate in Extremities, because this value mirrors the lasting lactate levels in the body and associated with defense mechanism of the body to drain the excess of lactate away from heart, brain and other organs. In the same manner as HbA₁C mirrors the lasting over the time levels of glucose. The value of lasting lactate had decreased in all measured persons (Table 16). The meaning of it could be explained as permanent decreased production or increased clearance of lactate in the body as response to the treatment with among others drinkable oxalates. All measurements were in compliance with the reported decrease in symptoms burden (FIG. 24) and fatigue presence (FIG. 25) and increased daily activity—CEDL (Tab. 22).

TABLE 16
Parameters from lactatograms before and under the treatment and under the placebo-test.

| | Capillary Lactate, mmol/L | | | Total Lactate Load, mmol/L*min, | | | Mean Lactate Level in | | |
|---|---|---|---|---|---|---|---|---|---|
| | (+/−S.D.), N = 26 | | | (AUC₁₅₅) | | | Extremities, N = 4 | | |
| | | 20 weeks | 7-18 days | | 20 weeks | | | 20 weeks | 7-18 days |
| | Before | with | after | Before | with | Placebo | Before | with | after |
| | Treatment | treatment | exchange | Treatment | treatment | test | Treatment | treatment | exchange |
| User 1 | 3.0 (1.0) | 1.92 (0.4) | 2.20 (0.8) | 464.8 | 303.0 | 351.8 | 8.4 (7.6) | n.d. | n.d. |
| User 3 | n.d. | n.d. | 1.80 (0.8) | n.d. | n.d. | 268.0 | n.d. | 1.7 (0.4) | 3.9 (3.1) |
| User 7 | 3.5 (1.8) | 2.24 (0.9) | 2.25 (0.9) | 537.7 | 345.0 | 344.0 | 5.3 (4.1) | 1.8 (0.4) | 2.3 (1.8) |
| User 8 | 3.5 (3.2) | 1.25 (0.4) | 1.44 (0.5) | 571.9 | 188.7 | 231.9 | 1.7 (0.6) | 1.1 (0.5) | 1.5 (0.1) |
| User 11 | 1.8 (0.8) | 2.1 (0.6) | 2.1 (0.6) | 268.2 | 311.2 | 325.2 | 2.3 (0.4) | 2.0 (0.8) | 2.0 (0.8) |
| User 12 | 1.8 (1.1) | 1.8 (0.4) | 1.6 (0.4) | 298.3 | 274.5 | 245.0 | 3.9 (2.7) | 2.1 (0.5) | 1.8 (0.7) |

### EXAMPLE 6—COGNITIVE RESTITUTION UNDER THE TREATMENT FOR CFS/ME/SEID AND HYPERLACTATEMIA

Introduction

The background for testing cognitive functioning was an interest from the subject herself She had recently been diagnosed with a condition Myalgic Encephalomyelitis (ME). She was already suffering from variety of the somatic symptoms (described above), when experienced the dramatic reduction in her cognitive abilities. The extremely and most dramatic worsening came fast; only within a few weeks she lost her ability to function normally on the daily basis. This reduction was of such degree and scope that she could not understand the 1-3 steps instructions for making a bag soup; loss ability to understand the foreign language she used on daily basis at work (English); could not find the words in her operative daily language (Norwegian); the logical practical operations such as order of operations during “pour a cap of a tea from the machine” was disrupted and reasoning of the order took extremely long time (some few seconds longer than she was used to). Moreover, she could not understand the words spoken to her and used few minutes to understand short sentences; was not able to perform the mathematical operations such addition and subtraction, the multiplication was forgotten for good. She could not perceive the spoken two-digits numbers. It seems that all cognitive operations took longer time than she was used to. All this together made her afraid of her health condition and cognitive functioning.

Unfortunately, the cognitive dysfunction is usual for whole group of the ME/SEID/CFS/Chronic hyperlactatemia affected patients and some of the mentioned above symptoms are on the List of the Canadian Criteria (2003), which is necessarily to fulfil to get the diagnosis (elsewhere).

The hypothesis was as following: The novel mix of niacin-riboflavin-thiamine-alpha-lipoic acid alone or in combination with drinkable mix of oxalates and carbohydrates can have a positive effect on cognitive functioning in persons affected by ME/SEID/CFS/Chronic hyperlactatemia.

Methods

At the assessment time the subject was 45 years old, she was on the sick leave from her work as a leader and researcher. On the other hand, she was optimistic due to possibility of recovering. CDL-90 test was performed before WAIS-IV test to exclude the depression or other psychological disorders. This test was negative on any disorders and it was decided to perform WAIS-IV test.

The subject was tested with WAIS-IV three times. First time full scale, and second and third time partially with subtests inside the working memory index and process speed index. The authorised phycologist performed the tests in his office.

The subject was aware of that energy level can affect the outcome of the tests and minimised the potential variations by doing the same routines at least for 3 days prior to testing, by keeping the same number of steps per day, scope of activities and their duration and nutritional diet before each assessment.

The subject, who was the same as P1 in rapport on self-treatment for ME/CFS/SEID/Chronic hyperlactatemia, was tested 3 times:
1.1.1.1 Assessment: Full scale WAIS-IV, date 28 Aug. 2014;
1.1.1.2 Assessment: Partial WAIS-IV, date 30 Sep. 2014;
1.1.1.3 Assessment: Partial WAIS-IV, date 5 Dec. 2014.

Following treatment was used at corresponding assessments:
a) 28 Aug. 2014 corresponds to period without treatment;
b) 30 Sep. 2014 corresponds to day 26 in a 33 days-long period with treatment by alpha-lipoic acid 300 mg once a day. Alpha-lipoic acid was taken 10:57 a.m.—right before the test started at 11:00 a.m. Additionally 250 ml of drinkable had been taken together with food at 10:42 a.m. This was a not routine, but at that time she used drinkable occasionally without understanding how to use it or why to use it. This was an exploratory and intuitive way to use it—on the demand of the body, with only signal—flu-like feeling and freezing.
c) 5 Dec. 2014 corresponds to result from 66 days with steadily increased dose of ALA, where last 29 days were with treatment by 1200 mg alpha-lipoic acid+12 mg thiamine+27 mg niacin+2.5 mg riboflavin per day in addition to two portions of drinkable, 250 ml each per day. On the test day 300 mg Alpha-lipoic acid was taken at 10:57 a.m., but the subject forgot to take the drinkable at 10:42 a.m. together with food as she did last time (assessment on 30 Sep. 2014).

Results

Assessment before treatment start—ME/CFS/SEID/Chronic Hyperlactatemia—28 Sep. 2014

Scores on the indexes shows functioning above average on all indexes. The full-scale score is not valid because of the huge discrepancy in scores between the indexes.

VCI=IQ 139, (99.5%)

PRI=IQ 126, (96%)

WMI=IQ 105, (63%)

FPSI=IQ=112, (79%)

PSIQ=126, (96%)

GAI=138 (99%)

In this score, we have to use the General Ability Index (GAI) which is very high and based on Verbal Comprehension (VCI) and Perceptual reasoning (PRI). Since the score on Working memory (WMI) and Process speed (PSI) is a lot lower than the others, it is reasonable to assume that the subjects experience of lower functioning than her normal state, is reflected in the scores on those two indexes. The discrepancy between index scores that makes it also reasonable to assume that the FSIQ would have been higher in her normal state, and that GAI best reflects her intellectual capacities without illness.

Assessment: Partial WAIS-IV, Date 30 Sep. 2014

The test was performed on 26^{th} day with 300 mg/day of Alpha-lipoic acid (ALA). The 300 mg ALA, 153 mg soluble oxalates with 25 g carbohydrates had been taken within 18 minutes prior to the test. The second test gives a valid result on Working memory and one subtest result on PSI and two subtest results on PRI: WMI=IQ 116 (86%). The results give significant higher score in working memory, and no valid differences in scores on process speed or Perceptual reasoning.

The perception of the second test was described by the subject in her diary: “Pictures which were used in the test were not that blinding as during the first assessment. The testing professional had assured me that the same lighting was used. That could mean that I became less light sensitive during this moths with the treatment. The problems with the vision was during both tests. However, it was much easier to percept the long rows of numbers, despite of it the boarder of how many numbers I could remember was very clear. I did not felt enough strength in the brain to remember all of them. Opposite to the first assessment I had strength in the brain, and not a porridge or black blanket as at first test. Despite of it, it was not enough that I could solved some mathematical assignments, simply because I could not perceive two-digits numbers during all assessments . . . ”

Assessment: Partial WAIS-IV, Date 5 Dec. 2014

The test was performed after 2 months, where last 29^{th} days were on the treatment consisting the novel mix of 1200 mg alpha-lipoic acid+12 mg thiamine+27 mg niacin+2.5 mg riboflavin per day in addition to two portions of drinkable, 250 ml each. On the test day, the only 300 mg ALA had been taken within 18 minutes, while the subject forgot to take the drinkable before the test. The third assessment gives a valid result on Working memory and one subtest on PSI and two subtest results on PRI: WMI=96 (39%). The scores give a significant loss in scores compared to first and second assessment. However, when we look in to the subtest scores, and look at the scores on the two main subtests administrated under all of the three assessments, the scores are basically the same with same variation.

Discussion and Conclusion

The WAIS-IV shows that the subject has a high level of cognitive functioning, with high IQ, but a significant lower functioning on the working memory index and the process speed index. These results are in accordance with her experienced symptoms and illness (ME/SEID/CFS/Chronic hyperlactatemia).

The increase of Working memory score by 23% from 63 to 86% measured on 26^{th} day of treatment most likely due to treatment with 300 mg ALA on the long term. However, at the test day, 18 minutes prior to test the person had taken both the ALA and oxalates. General knowledge is that ALA absorbs into the blood stream within 30 min and excreted via urine within 30-60 min post intake. This is in compliance of the time for noticeable therapeutic effect in ME/SFC/SEID patients who use our self-treatment (described elsewhere). However, ALA is both water- and fat-soluble and therefore its pharmacokinetics have to be more complicated and affected by body fat index. On another hand, soluble oxalates are only water soluble. Our numerous tests on healthy and ME affected persons had confirmed that effect is noticeable already within 10 min, with Tₘₐₓ of 102-150 min post intake and effect duration for 4.5-5 hours. Therefore, it is reasonable to say that maximum effect of oxalate coincidence with the test period. Therefore, it is not possible to exclude oxalate as a potential candidate responsible for the achieved therapeutic effect.

This assumption could be also deducted from the results of the test on 5 Dec. 2014, when treatment was extended by new compounds and increase dose of ALA from 300 to 1200 mg a day when it comes to the long-term effect of the treatment. However, at that test day the person had taken only 300 mg ALA, e.g. the same amount as at the day of the second assessment and did not taken 250 ml of drinkable with 153 mg soluble oxalates and 25 g mixture of carbohydrates. Therefore, if we were looking away from the long-term effect and assume that only treatment taken before the test would affect the outcome, then the difference lies only in absence/presence of soluble oxalates in a treatment taken before the test.

Working Memory score increased by 23% on the test day 30 Sep. 2014, when oxalates were taken, and opposite, when oxalates where not taken, the decrease in Working Memory score by 47% on the test 5 Dec. 2014 were documented.

From this day, the extremely importance of the oxalates for cognitive functioning had been discovered. The treatment had been adjusted in such way that systematic supply of soluble oxalates along with carbohydrate mix was combined with systematic supply of novel formulation of alpha-lipoic acid-niacin-thiamine-riboflavin during a day.

Today, the test person is fully functioning and has none cognitive difficulties experienced before the treatment. She is able to concentrate herself for many hours, as it was before her illness. We suggest including the soluble oxalates on the list of chemical compounds with ability to improve cognitive functioning and cognitive restitution. However, we aware that test on several persons should be performed. On another hand, 11 persons affected with ME/SEID/CFS/Chronic hyperlactatemia who uses the self-treatment had reported improvement of their cognitive functioning, among other: exertional malaise, including abnormal loss of mental endurance, rapid mental/cognitive fatigability, and abnormal long recovery; post exertional malaise or fatigue; post exertional disease worsening; impaired short-term memory and concentration; difficulty in processing information, finding words —all from the list of Canadian Criteria (2003). In addition, they also reported less severity of the cognitive symptoms associated with fatigue, such as: concentration problems; problems to think clearly; memory problems; difficulties to find the words. The reduction in symptoms and reduction of fatigue presence corresponds to the results achieved by treatment and as documented also applies to the cognitive restitution of the ME/CFS/SEID/Chronic Hyperlactatemia patients.

The next evidence of that oxalates plays a role in cognitive recovery of ME/CFS/SEID/Chronic hyperlactatemia patients would be the results from test on lowering oxalate dose (placebo-exclusion test). FIG. 25 shows fatigue presence at 20 or 110 weeks on the same treatment as described for assessment 3 in this report; three days on the same treatment, including 610 mg soluble oxalates per day along with carbohydrate mix and vitamin-ALA mix; 5 to 22 days on lowered by 60% dose of soluble oxalates, carbohydrate mix and vitamin-Ala mix; and again 5 to 22 days on 610 mg oxalates, carbohydrates and vitamin-ALA mix. Thus, amount of carbohydrates and novel formulation of ALA with vitamins were not affected by lowering oxalate dose (Tab. 1.) in the Placebo-test, they will be not discussed.

The overall trend for the 6 tested persons with ME/SEID/CFS/Chronic hyperlactatemia shown on FIG. 26. There were 11 questions in the questionnaire “Fatigue Scale/Presence, FS”, where 4 of them were addressed difficulties in cognitive functioning. Minimum points on this schema was 0 and maximum 33, where 0-12 points were accounted for cognitive functioning. For example, P11 had reported mean score of 9 after 20 weeks with 610 mg oxalates/day and increased to 28 within 14 days on lower dose of 270 mg/day. The increase in score was due to corresponding increase from 4 to 8 points for cognitive functioning on 4^{th} day and from 8 to 9 during 5^{th} day and finally to maximum 12 points after 1 week on reduced dose of oxalates. The same was truth for 5 of 6 persons tested: the person P7 did not have any fluctuations in mentioned symptoms during this test.

On the long term, all 11 persons had reported improvement in their cognitive functioning: P3 and P4 spending now about 4 hours a day of exercising mathematics and cross-words. P8 reading a lot and able to remember better; P12 working as a teacher at 20%; P1 can work for 10 hours some days with writing; P2 and P5 reported that their thoughts became faster and clear. P9 is only in the beginning of the recovery and did not experienced much difference yet.

Conclusions
The soluble oxalates were documented to improve the Working memory score according to WAIS-IV assessment when used in combination to alpha-lipoic acid. However, the alpha-lipoic acid alone did not have the same therapeutic effect. Therefore, the achieved effect was credited to the soluble oxalates.
To maintain the long-term therapeutic effect on cognitive functioning, the systematic supply of at least 610 mg soluble oxalates per day should be assured in mandatory combination with carbohydrate mix and combination to optional dose of novel formulation of alpha-lipoic acid+thiamine+niacin+riboflavin per day.
The soluble oxalates had been shown to play key-role in the cognitive recovery of the ME/CFS/SEID/Chronic Hyperlactatemia patients supplied with described above treatment on the long-term basis.
The therapeutic effect was demonstrated to be dose-dependent and thus could not be due to placebo-effect.
The cognitive functioning is very vulnerable to the absence of the treatment and symptoms had shown reversible nature and appears/disappears in the same order in the same person, each time treatment stops or starts.
We claim that oxalates can be used for cognitive restitution, recovery and maintenance of normal cognitive functioning in conditions associated with hyperlactatemia or not.

### EXAMPLE 7—IMPROVEMENT OF HEALTH AS DIMINISHED SYMPTOMS FOR THE LIST OF CANADIAN CRITERIA

A representative example of the composition that has been used in the testing was given as following: during first 20 weeks, the dosage was gradually and systematically increased in accordance with the progression and perception of the patient. From week 21 all patients received equal medication as shown above. After all it was nessesarily reduce the dosage of thiamine, which explains the lower dose from week 21.

From week 0 to 20 the dosage was gradually and systematically increased: 300-1200 mg alpha-lipoic acid; 3-12 mg thiamine; 27-81 niacin per day and riboflavin—0-0.31 mg per week. From week 21: 1200 mg alpha-lipoic acid; 9 mg thiamine; 81 mg niacin per day and riboflavin 0.31 mg per week were used for self-treatment. Daily dose of oxalates in Active drinkable was as described in Example 1, 610-710 mg and was the same though all reported period.

Table shows some of the results obtained—reduced number of symptoms within given time with treatment.

TABLE 23
Results of the treatment during given time on reduction of symptoms
from the List of Canadian Criteria for P1, P2 and P3.

| | Patient 1 | | Patient 2 | | Patient 3 | |
|---|---|---|---|---|---|---|
| Weeks with the treatment | 0 | 48 | 0 | 65 | 0 | 91 |
| 1. Fatigue | X | − | X | − | X | − |
| 2. Exertional malaise | | | | | | |
| a) Abnormal loss of physical and | X | − | X | +/− | X | − |
| mental endurance. Rapid muscular | | | | | | |
| and mental/cognitive fatigability. | | | | | | |
| Malaise and fatigueor/and pain after | | | | | | |
| load, and abnormal long recovery. | | | | | | |
| b) Post exertional malaise or fatigue | X | − | X | − | X | − |
| c) Post exertional disease worsening | X | − | X | − | X | − |
| 3. Sleep disturbances (Table 1-2) | X | − | X | X | X | − |
| 4. Pain | X | − | X | X | X | − |
| 5. Neurological or cognitive symptoms | | | | | | |
| a) Impaired short-term memory | X | − | X | X | X | − |
| and concentration | | | | | | |
| b) Difficulty in processing information, | X | − | X | X | X | − |
| finding words and periodically | | | | | | |
| speech problems | | | | | | |
| c) Disorientation and confusion | X | − | X | — | X | − |
| d) Diffuculty in finding words | X | − | X | X | X | − |
| e) Muscle weakness and muscle twitching | X | − | X | − | X | − |
| f) Dizziness and balance problems | X | − | X | − | X | − |
| g) Sensory disturbances, difficulty | X | − | X | − | X | − |
| focusing vision, numbness/coldness | | | | | | |
| h) Hypersensitivity to light, sound and | X | − | X | +/− | X | − |
| stress, which can lead to a crash | | | | | | |
| periods and or anxiety | | | | | | |
| 6A. Autonomous symptoms | | | | | | |
| a) Orthostatic intolerance | X | − | X | − | X | − |
| b) Palpitations (tachycardia) | X | − | X | − | X | − |
| c) Irritable Bowie Syndrome, IBS | X | − | X | − | X | X |
| d) Difficulty breathing | X | − | X | − | X | − |
| e) Frequent nocturnal urination | X | − | X | − | X | − |
| 6B. Neuroendocrine symptoms | | | | | | |
| a) Altered temperature | X | − | X | − | X | − |
| regulation/night sweats | | | | | | |
| b) Heat/cold intolerance | X | − | X | − | X | X |
| c) Anorexia or abnormal appetite | X | − | X | − | X | − |
| 6C. Immunological symptoms | | | | | | |
| a) Tender lymph nodes on the | X | − | X | − | X | − |
| neck/under arm holes | | | | | | |
| b) Periodically sore throat | X | − | X | − | X | − |
| c) Flu-like symptoms and malaise | X | − | X | X | X | − |
| d) Development of new | X | − | X | − | X | − |
| allergic reactions | | | | | | |
| e) Hypersensitivity for medicine | X | − | X | − | X | − |
| and/or chemicals | | | | | | |
| 7. Symptom stream | | | | | | |
| a) Condition lasted for at | X | X | X | X | X | X |
| least 6 months | | | | | | |
| b) Fatigue started with well defined | X | − | − | − | − | − |
| debut within few weeks | | | | | | |
| c) Fatigue developed gradually | X | X | X | X | X | X |
| d) Fatigue and symptoms are | X | X | − | X | − | X |
| stable or improving | | | | | | |
| e) Fatigue and symptoms are | X | − | X | − | X | − |
| gradually worsened | | | | | | |

We have also conducted the same experiment as described above, but without Using Active drinkable, and the results show no effect. The same was also documented by WAIS-IV test (Example 6). We can thus conclude that oxalic acid is one of the central and essential compounds of the treatment composition and achievement of the results shown in Tab. X is impossible without it.

Administration of the Composition of the Present Invention

As a pharmaceutical medicament the compositions of the present invention may be administered directly to the patient or mammal by any suitable technique, including parenterally, intranasally, orally, or by absorption through the skin. They can be administered locally or systemically. The specific route of administration of each agent will depend, e.g., on the medical history of the patient.

Examples of parenteral administration include subcutaneous, intramuscular, intravenous, intraarterial, and intraperitoneal administration.

For parenteral administration, in one embodiment, the compositions of the present invention are formulated generally by mixing each at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation.

For oral pharmacological compositions such carrier material as, for example, water, gelatine, gums, lactose, starches, magnesium-stearate, talc, oils, polyalkene glycol, petroleum jelly and the like may be used. Such pharmaceutical preparation may be in unit dosage form and may additionally contain other therapeutically valuable substances or conventional pharmaceutical adjuvants such as preservatives, stabilising agents, emulsifiers, buffers and the like. The pharmaceutical preparations may be in conventional liquid forms such as tablets, capsules, dragees, ampoules, drinkable solutions and the like, in conventional dosage forms.

Appendix 1. Overview of the Medicinal Drug Use by Each User Before and in Combination with this Treatment.

Three persons were not receiving the drinkable for self-treatment: 2, 5 and 6. Person 5 was excluded after 20 weeks because of the use of Escitalopram Oxalate (with indication to depressive disorder and generalized anxiety disorder), which gave massive diarrhea in combination with mixture of drinkable oxalates. Person 6 was excluded of toxicological consideration after 20 weeks because massive use of painkiller Paralgine forte, 600 mg: 20 tablets per day, and was practically an abuser of medicinal drugs. Person 2 was excluded because use of sleep medicine—Alimemazin tartrate. All three drugs listed with long list of the negative by-effects. Those effects are very similar to the symptoms on the list of Canadian Criteria. Further treatment with mixture of drinkable oxalates was therefore is not considerate.

List of medicinal drugs used and prescribed to ME affected and which were successfully rejected by users before treatment start. Nobody get back to use them again, because the symptoms had disappeared as result of their self-treatment according to given schema and compounds.

There are: Maxalt Rapitab, Adalat Oros, Valsartan, Esidrex, Imovane, Truxal, Melatonin, Quetiapin, Metropolol, Cipralex, Paralgin Forte, Adalat Oros, Cyclofosfamid, Rituximab, to mention some.

Now the person who used Escitalopram Oxalate try the treatment once more time. Dose of the soluble oxalates in Active drinkable will be reduced accordingly to dose of Escitalopram Oxalate. In this way the daily dose of soluble oxalates will not exceed the dose of 610-710 mg.

### REFERENCES

1. Honow, R., & Hesse, A. (2002). Comparison of extraction methods for the determination of soluble and total oxalate in foods by HPLC-enzyme-reactor. Food Chemistry, 78, 511-521.
2. Al-Wahsh, I. A., Wu, Y., & Liebman, M. (2012). A Comparison of Two Extraction Methods for Food Oxalate Assessment., Vol. 1 No. 2, 233-237.
3. Da Costa, L. M., Tronto, J., Constantino, V. R. et al. (2009). Extraction and concentration of biogenic calcium oxalate from plant leaves. Nota. R. Bras. Ci. Solo., 33:729-733.

1.-26. (canceled)

27. A composition for use in the prevention and/or treatment of chronic fatigue syndrome (CFS)/myalgic encephalomyelitis (ME)/systemic exertion intolerance disease (SEID), comprising administering to a patient in need thereof a pharmaceutical or nutritional composition comprising oxalate or oxalic acid, or a derivate or salt thereof, wherein the derivate is oxaloacetic acid.

28. The composition for use according to claim 27, wherein said composition comprises an oxalic compound of the formula 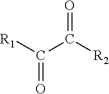
where;
R₁═OH and R₂═OH is oxalic acid, or,
or
R₁═O⁻ or R₂═OH is hydrogen oxalate, or
R₁═K⁺O⁻, R₂═OH is potassium hydrogenoxalate, or
R₁═O⁻ and R₂═O⁻ is oxalate, or
R₁═NaO and R₂═NaO is sodium oxalate, or
R₁═CaO and R₂═CaO is calcium oxalate

29. The composition for use according to claim 27, comprising a compound selected from the group consisting of magnesium oxalate, potassium oxalate, oxalic acid anhydrous, oxalic acid dihydrate, lithium oxalate, cesium oxalate, lithium oxamate, caesium oxalate, beryllium oxalate, potassium oxalate, oxalic acid anhydrous, lithium oxalate, sodium oxalate, thallium (I) oxalate, uranyl oxalate, gallium oxalate, gold oxalate, magnesium oxalate, mercury (II) oxalate, manganese oxalate, nickel oxalate, barium oxalate, silver oxalate, iron (II) ferrous oxalate, scandium oxalate, cadmium oxalate, and calcium oxalate.

30. The composition for use according to claim 27, wherein the composition also comprises lipoic acid, preferably alpha-lipoic acid (ALA).

31. The composition for use according to claim 27, wherein the composition also comprises thiamine, B₁.

32. The composition for use according to claim 27, wherein the composition also comprises niacin, B₃.

33. The composition for use according to claim 27, wherein the composition also comprises Riboflavin, B₂.

34. The composition for use according to claim 27, wherein the composition also comprises a sugar.

35. The composition for use according to claim 34, wherein the sugar is wherein the sugar is one or more sugars selected from the group consisting of sucrose, glucose, fructose, maltose and lactose.

36. The composition for use according to claim 27, wherein the patient has an additional disorder, wherein the additional disorder comprises one or more symptoms or disorders selected from, hyperlactataemia, acidosis, myalgic encephalomyelitis (ME), fibromyalgia, ageing, sleep quality, and physical and cognitive activity.

37. The composition for use according to claim 27, wherein the patient has an additional disorder, wherein the additional disorder or disease is selected from;
i) Heart functioning failure as acute cardiac patients with acute coronary syndrome, cardiogenic shock, cardiac arrest with hyperlactemia (high lactate levels and poore lactate clearance), high resting heart rate.
ii) Ortostatic intolerance, essential hypertention, hypotention
iii) All metabolic disorders where occumulation of lactate and its delated or poore excretion/clearance are main cause of the death;
iv) All normal physiological and medical conditions where occumulation of lactate and its delated or poor excretion/clearance are present;
v) All cognitive disorders, which are caused by or causing the occumulation of lactate and its delated or poor excretion/clearance;
vi) All medical conditions where functioning of the organs, tissues and systems are corrupted as the result of the decreased energy production and occumulation of lactate and its delated or poor excretion/clearance as listed in pkt. 1-5.
vii) As claimed in pkt. 6 and in addition to mentioned all symptoms in Tab.3 also astma provoked by physical activity (extreme sports) and/or cold.
viii) Sleep disturbance, especially insomnia
ix) Irritable Bowel syndrome
x) Fatigue caused by chemotherapy.
